(19) United States
(12) Patent Application Publication
(10) Pub. No.: US 2005/0113413 A1
(43) Pub. Date: May 26, 2005

(54) Naphthalene-1,5-disulfonic acid salts of a substituted 4-amino-1-(pyridylmethyl)piperidine compound

(76) Inventors: Richard D. Wilson, El Sobrante, CA (US); Julie Congdon, San Francisco, CA (US); Mathai Mammen, Redwood Shores, CA (US); Weijiang Zhang, Concord, CA (US); Robert Chao, Santa Clara, CA (US)

Correspondence Address: THERAVANCE, INC., 901 GATEWAY BOULEVARD, SOUTH SAN FRANCISCO, CA, 94080, US

(21) Appl. No.: 10/975,657

(22) Filed: Oct. 28, 2004

Related U.S. Application Data
(60) Provisional application No. 60/515,394, filed on Oct. 29, 2003.

(51) Int. Cl. A61K 31/4545; C07D 41/14

(52) U.S. Cl. 514/316; 546/193

(57) ABSTRACT

This invention provides naphthalene-1,5-disulfonic acid salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine, which salts are useful as muscarinic receptor antagonists. This invention is also directed to pharmaceutical compositions comprising these salt forms, methods of using these salt forms for treating medical conditions mediated by muscarinic receptors; and processes for preparing these salt forms.

### CROSS-REFERENCE TO RELATED APPLICATIONS

This application claims the benefit of U.S. Provisional Application No. 60/515,394, filed on Oct. 29, 2003, which application is incorporated herein by reference in its entirety.

### BACKGROUND OF THE INVENTION

1. Field of the Invention

This invention is directed to naphthalene-1,5-disulfonic acid salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine, which salts are useful as muscarinic receptor antagonists. This invention is also directed to pharmaceutical compositions comprising such salt forms, methods of using such salt forms for treating medical conditions mediated by muscarinic receptors; and processes for preparing such salt forms.

2. State of the Art

Muscarinic receptor antagonists are useful for treating various medical conditions mediated by muscarinic receptors, such as overactive bladder (OAB), irritable bowel syndrome (IBS), asthma and chronic obstructive pulmonary disease (COPD). Commonly-assigned U.S. Provisional Application Nos. 60/422,229, filed on Oct. 30, 2002; and 60/486,483, filed on Jul. 11, 2003; and U.S. patent application Ser. No. 10/696,464, filed on Oct. 29, 2003; disclose novel substituted 4-amino-1-(pyridylmethyl)piperidine and related compounds that are useful as muscarinic receptor antagonists. In particular, the compound, 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine is specifically disclosed in these applications as an effective muscarinic receptor antagonist.

The chemical structure of 4-{N-[7-(3-(S)-1-carbamoyl-1,1-diphenylmethyl)-pyrrolidin-1-yl)hept-1-yl]-N-(isopropyl)amino}-1-(4-methoxypyrid-3-ylmethyl)piperidine is represented by formula I:

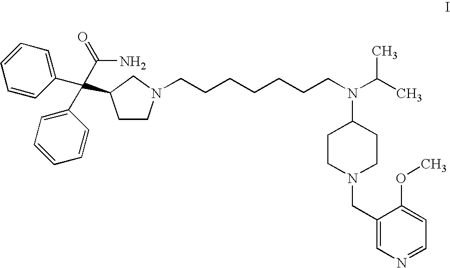

To effectively use this compound as a therapeutic agent, it would be desirable to have a salt form that can be readily manufactured and that has acceptable chemical and physical stability. For example, it would be highly desirable to have a salt form that minimizes the formation of impurities during the preparation and subsequent storage of the salt. Additionally, the salt form should have acceptable hygroscopicity, i.e., it should remain a free flowing powder and not be deliquescent when exposed to atmospheric moisture. No such salt forms have previously been reported. Accordingly, a need exists for a stable, non-deliquescent salt form of the compound of formula I.

### SUMMARY OF THE INVENTION

The present invention provides naphthalene-1,5-disulfonic acid salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine, which are useful as muscarinic receptor antagonists. In the salts of this invention, the molar ratio or stoichometry of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.7 to about 1.1.

Unlike other salt forms of this compound, the naphthalene-1,5-disulfonic acid salts of this invention have been discovered not to generate significant amounts of undesired impurities during formation and subsequent storage of the salt. Additionally, unlike other salt forms, the naphthalene-1,5-disulfonic acid salts of this invention have been found to have acceptable hygroscopicity and not to be deliquescent when exposed to atmospheric moisture.

Accordingly, in one of its composition aspects, this invention provides a naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or a solvate thereof; wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.7 to about 1.1. In a particular embodiment of this aspect of this invention, the salt form is an amorphous powder.

In another of its composition aspects, this invention provides a pharmaceutical composition comprising a pharmaceutically-acceptable carrier and a 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt of this invention.

The compound of formula I is a muscarinic receptor antagonist. Accordingly, in one of its method aspects, this invention provides a method for treating a medical condition alleviated by treatment with a muscarinic receptor antagonist in a mammal, the method comprising administering to the mammal a therapeutically effective amount of a pharmaceutical composition comprising a pharmaceutically-acceptable carrier and a 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt of this invention.

In another of its method aspects, this invention provides a method for treating overactive bladder in a mammal, the method comprising administering to the mammal a therapeutically effective amount of a pharmaceutical composition comprising a pharmaceutically-acceptable carrier and a 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt of this invention.

This invention is also directed to processes for preparing the naphthalene-1,5-disulfonic acid salts of the compound of formula I. Accordingly, in another of its method aspects, this invention provides a process for preparing a naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine; the process comprising contacting 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine with about 0.7 to about 1.1 molar equivalents of 1,5-naphthalenedisulfonic acid or a hydrate thereof.

This invention is also directed to a 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt of this invention for use in therapy or as a medicament.

Additionally, this invention is directed to the use of a 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt of this invention for the manufacture of a medicament; especially for the manufacture of a medicament for the treatment of a medical condition which is alleviated by treatment with a muscarinic receptor antagonist, such as overactive bladder.

### DETAILED DESCRIPTION OF THE INVENTION

This invention provides certain naphthalene-1,5-disulfonic acid salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine. The active agent in these salts (i.e., the compound of formula I) contains one chiral center having the (S) configuration. However, it will be understood by those skilled in the art that minor amounts of the (R) stereoisomer may be present in the compositions of this invention unless otherwise indicated, provided that the utility of the composition as a whole is not eliminated by the presence of such an isomer.

If desired, alternative nomenclature may be used to described the compound of formula I and its naphthalene-1,5-disulfonic acid salts. For example, the compound of formula I can also be named using AutoNom (MDL, San Leandro Calif.) as follows: 2-[(S)-1-(7-{isopropyl-[1-(4-methoxypyridin-3-ylmethyl)piperidin-4-yl]amino}heptyl)pyrrolidin-3-yl}-2,2-diphenylacetamide. Additionally, naphthalene-1,5-disulfonic acid salts are also known as napadisylate salts.

Definitions

When describing the compounds, compositions, methods and processes of this invention, the following terms have the following meanings unless otherwise indicated.

The term “overactive bladder” or “OAB” refers to a condition characterized symptomatically by urinary urge, urinary incontinence, increased frequency of urination, and/or nightime urination and the like. The term “urinary urge” refers to a strong and sudden desire to void the bladder.

The term “solvate” refers to a complex or aggregate formed by one or more molecules of a solute, i.e. a compound of this invention, and one or more molecules of a solvent. Representative solvents include, by way of example, water, methanol, ethanol, isopropanol, acetic acid and the like. When the solvent is water, the solvate formed is a hydrate.

The term “therapeutically effective amount” refers to an amount sufficient to effect treatment when administered to a patient in need of treatment.

The term “treating” or “treatment” as used herein refers to the treating or treatment of a disease or medical condition (such as overactive bladder) in a patient, such as a mammal (particularly a human or a companion animal) which includes:
(a) preventing the disease or medical condition from occurring, i.e., prophylactic treatment of a patient;
(b) ameliorating the disease or medical condition, i.e., eliminating or causing regression of the disease or medical condition in a patient;
(c) suppressing the disease or medical condition, i.e., slowing or arresting the development of the disease or medical condition in a patient; or
(d) alleviating the symptoms of the disease or medical condition in a patient.

The term “unit dosage form” refers to a physically discrete unit suitable for dosing a patient, i.e., each unit containing a predetermined quantity of the salt of the invention calculated to produce the desired therapeutic effect either alone or in combination with one or more additional units. For example, such unit dosage forms may be capsules, tablets, pills, and the like.

### Naphthalene-1 5-disulfonic Acid Salts of the Invention

The 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention can be prepared from 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine and naphthalene-1,5-disulfonic acid or a hydrate thereof.

In the salts of this invention, the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.7 to about 1.1; including about 0.8 to about 1.05; and about 0.9 to about 1. Other ranges for the molar ratio include about 0.7 to about 1.05; about 0.7 to about 1; about 0.7 to about 0.95; about 0.8 to about 1.1; about 0.8 to about 1; about 0.8 to about 0.95; about 0.9 to about 1.1; about 0.9 to about 1.05, about 0.9 to about 0.95; about 0.95 to about 1.05; and about 0.95 to about 1.

The molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine can be readily determined by various methods available to those skilled in the art. For example, such molar ratios can be determined by ¹H NMR. When using ¹H NMR, the molar ratio is typically determined by comparing the integration for the naphthalene ring protons of the naphthalene-1,5-disulfonic acid to the integration for the pyridine ring protons in the compound of formula I. Alternatively, elemental analysis and HPLC methods can be used to determine the molar ratio.

The 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine employed in this invention can be readily prepared from commercially available starting materials and reagents using the procedures described in the Examples below; or using the procedures described in the commonly-assigned U.S. applications described in the Background section of this application.

Naphthalene-1,5-disulfonic acid (also known as Armstrong's Acid) is commercially available from, for example, Aldrich, Milwaukee, Wis. In one embodiment, the naphthalene-1,5-disulfonic acid employed in this invention is a hydrate, such as the tetrahydrate.

To prepare the salts of this invention, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine is typically contacted with about 0.7 to about 1.1 molar equivalents of naphthalene-1,5-disulfonic acid or a hydrate thereof. Generally, this reaction is conducted in an inert diluent at a temperature ranging from about −20° C. to about 40° C.; including about 0° C. to about 20° C., such as about 2° C. to about 15° C. Suitable inert diluents for this reaction include, but are not limited to, methanol, ethanol, isopropanol, isobutanol, ethyl acetate and the like.

Upon completion of the reaction, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt is isolated from the reaction mixture by any conventional means, such as precipitation, concentration, centrifugation and the like.

In one embodiment, the salts of this invention are an amorphous powder. Such amorphous powders are typically prepared by (1) forming a solution of the salt in a first inert diluent in which the salt is readily soluble (i.e., typically having a solubility greater than about 50 mg/mL); and then (2) contacting this solution with a second inert diluent (which can be a combination of inert diluents) in which the salt has lower or no solubility (i.e., typically having a solubility less than about 1 mg/mL), to form a precipitate.

Suitable first inert diluents for forming a solution of the salt include, but are not limited to, methanol, ethanol, isopropanol and the like, or combinations thereof. Generally, the salt is dissolved in the minimum amount of the first inert diluent necessary to form an essentially homogeneous solution.

Suitable second inert diluents for precipitating the salt include, but are not limited to, methy tert-butyl ether, isopropyl acetate and the like, or combinations thereof with isopropanol. In one embodiment, a 2:1 v/v mixture of isopropanol and methyl tert-butyl ether is employed as the second inert diluent.

If desired, the solution of the salt in the first inert diluent can be treated with activated carbon prior to adding the solution to the second inert diluent. Typically, the activated carbon is added to the solution and the resulting mixture is mixed, stirred or agitated for about 0.5 to about 2 hours at a temperature ranging from 0° C. to about 30° C. The mixture is then filtered to remove the activated carbon and any other insoluble materials that may be present.

To form the amorphous powder, a solution of the salt dissolved in a the first inert diluent is typically added slowly to the second inert diluent to form a precipitate. This process is typically conducted at a temperature ranging from about 0° C. to about 10° C.; such as about 2° C. to about 8° C. The rate of addition typically ranges from about 50 mL/minute to about 70 mL/minute for a solution containing about 0.20 g/mL to about 0.40 g/mL of the salt to be precipitated.

After formation, the precipitate is isolated using conventional procedures, such as filtration and the like, to provide the amorphous powder. If desired, the precipitate can be washed with an inert diluent, such as methyl tert-butyl ether, and then throughly dried.

Among other properties, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention have been discovered to have unexpected and surprising chemical and physical stability compared to other salt forms of this compound. In this regard, certain salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine have been found to be prone to chemical decomposition resulting in the formation of impurities. For example, two impurities detected in certain salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine are a 3-[4-{N-[7-(3-(S)-1-carbamoyl-1,1-diphenylmethyl)pyrrolidin-1-yl)hept-1-yl]-N-(isopropyl)amino}piperidin-1-ylmethyl]-4-methoxy-1-methylpyridinium salt (Impurity A) and 4-{N-[7-(3-(S)-1-carbamoyl-1,1-diphenylmethyl)pyrrolidin-1-yl)hept-1-yl]-N-(isopropyl)amino}-1-(4-oxo-1,4-dihydropyrid-3-ylmethyl)piperidine (Impurity B).

Surprisingly, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention have been found not to form significant amounts of Impurities A or B upon formation or prolonged storage of the salt. Accordingly, the compositions of this invention will typically contain less than 0.2 wt. %, including less than 0.1 wt. %, of Impurity A or B or both. In one embodiment, the compositions of this invention are essentially free of Impurity A or B or both, i.e., these impurities are below the limit of quantitation using standard analytical methods, such as HPLC.

Additionally, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention have been discovered to have unexpected and surprising physical stability when exposed to atmosphere moisture. In particular, the salts of this invention have been found not to be deliquescent and to remain a free flowing powder when exposed to atmospheric moisture. For example, when stored at 30° C. and 60% relative humidity for 15 days, salts of this invention remained a free-flowing powder. In contrast, other salts such as the di- and trimesylate salts absorbed water to form semi-solids or oils under the same storage conditions.

These properties of the salts of this invention are further illustrated in the Examples below.

Pharmaceutical Compositions

The 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention are typically administered to a patient in the form of a pharmaceutical composition. Such pharmaceutical compositions may be administered to the patient by any acceptable route of administration including, but not limited to, oral, rectal, vaginal, nasal, inhaled, topical (including transdermal) and parenteral modes of administration.

Accordingly, in one of its compositions aspects, this invention is directed to a pharmaceutical composition comprising a pharmaceutically-acceptable carrier or excipient and a therapeutically effective amount of a 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salt of this invention. Optionally, such pharmaceutical compositions may contain other therapeutic and/or formulating agents if desired.

The pharmaceutical compositions of this invention typically contain a therapeutically effective amount of a salt of this invention (i.e., the active agent). Typically, such pharmaceutical compositions will contain from about 0.1 to about 95% by weight of the active agent; preferably, from about 5 to about 70% by weight; and more preferably from about 10 to about 60% by weight of the active agent.

Any conventional carrier or excipient may be used in the pharmaceutical compositions of this invention. The choice of a particular carrier or excipient, or combinations of carriers or exipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially-available from, for example, Sigma, P.O. Box 14508, St. Louis, Mo. 63178. By way of further illustration, conventional formulation techniques are described in Remington: The Science and Practice of Pharmacy, 20^{th} Edition, Lippincott Williams & White, Baltimore, Md. (2000); and H. C. Ansel et al., Pharmaceutical Dosage Forms and Drug Delivery Systems, 7^{th} Edition, Lippincott Williams & White, Baltimore, Md. (1999).

Representative examples of materials which can serve as pharmaceutically acceptable carriers include, but are not limited to, the following: (1) sugars, such as lactose, glucose and sucrose; (2) starches, such as corn starch and potato starch; (3) cellulose, such as microcrystalline cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The pharmaceutical compositions of this invention are typically prepared by throughly and intimately mixing or blending a compound of the invention with a pharmaceutically-acceptable carrier and one or more optional ingredients. If necessary or desired, the resulting uniformly blended mixture can then be shaped or loaded into tablets, capsules, pills and the like using conventional procedures and equipment.

In a one embodiment, the pharmaceutical compositions of this invention are suitable for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present invention as an active ingredient.

When intended for oral administration in a solid dosage form (i.e., as capsules, tablets, pills and the like), the pharmaceutical compositions of this invention will typically comprise a compound of the present invention as the active ingredient and one or more pharmaceutically-acceptable carriers, such as sodium citrate or dicalcium phosphate. Optionally or alternatively, such solid dosage forms may also comprise: (1) fillers or extenders, such as starches, microcrystalline cellulose, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and/or sodium carbonate; (5) solution retarding agents, such as paraffin; (6) absorption accelerators, such as quaternary ammonium compounds; (7) wetting agents, such as cetyl alcohol and/or glycerol monostearate; (8) absorbents, such as kaolin and/or bentonite clay; (9) lubricants, such as talc, calcium stearate, magnesium stearate, solid polyethylene glycols, sodium lauryl sulfate, and/or mixtures thereof; (10) coloring agents; and (11) buffering agents.

Release agents, wetting agents, coating agents, sweetening, flavoring and perfuming agents, preservatives and antioxidants can also be present in the pharmaceutical compositions of this invention. Examples of pharmaceutically-acceptable antioxidants include: (1) water-soluble antioxidants, such as ascorbic acid, cysteine hydrochloride, sodium bisulfate, sodium metabisulfate sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like. Coating agents for tablets, capsules, pills and like, include those used for enteric coatings, such as cellulose acetate phthalate (CAP), polyvinyl acetate phthalate (PVAP), hydroxypropyl methylcellulose phthalate, methacrylic acid-methacrylic acid ester copolymers, cellulose acetate trimellitate (CAT), carboxymethyl ethyl cellulose (CMEC), hydroxypropyl methyl cellulose acetate succinate (HPMCAS), and the like.

If desired, the pharmaceutical compositions of the present invention may also be formulated to provide slow or controlled release of the active ingredient using, by way of example, hydroxypropyl methyl cellulose in varying proportions; or other polymer matrices, liposomes and/or microspheres.

In addition, the pharmaceutical compositions of the present invention may optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Solid dosage forms for oral administration of the pharmaceutical compositions of this invention are preferably packaged in a unit dosage form, including capsules, tablets, pills, and the like.

Suitable liquid dosage forms for oral administration include, by way of illustration, pharmaceutically-acceptable emulsions, microemulsions, solutions, suspensions, syrups and elixirs. Such liquid dosage forms typically comprise the active ingredient and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active ingredient, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In another embodiment, the pharmaceutical compositions of this invention are suitable for inhaled administration. Suitable pharmaceutical compositions for inhaled administration will typically be in the form of an aerosol or a powder. Such compositions are generally administered using well-known delivery devices, such as a metered-dose inhaler, a dry powder inhaler, a nebulizer or a similar delivery device.

When administered by inhalation using a pressurized container, the pharmaceutical compositions of this invention will typically comprise the active ingredient and a suitable propellant, such as dichlorodifluoromethane, trichlorofluoromethane, dichlorotetrafluoroethane, carbon dioxide or other suitable gas.

Additionally, the pharmaceutical composition may be in the form of a capsule or cartridge (made, for example, from gelatin) comprising a compound of this invention and a powder suitable for use in a powder inhaler. Suitable powder bases include, by way of example, lactose or starch.

The compounds of this invention can also be administered transdermally using known transdermal delivery systems and excipents. For example, a compound of this invention can be admixed with permeation enhancers, such as propylene glycol, polyethylene glycolm monolaurate, azacycloalkan-2-ones and the like, and incorporated into a patch or similar delivery system. Additional excipients including gelling agents, emulsifiers and buffers, may be used in such transdermal compositions if desired.

If desired, the pharmaceutical compositions of this invention may also contain other therapeutic agents that are co-administered with a salt of this invention. For example, the pharmacuetical compositions of this invention may further comprise one or more therapeutic agents selected from the group consisting of β₂ adrenergic receptor agonists, anti-inflammatory agents (e.g. corticosteroids and non-steroidal anti-inflammatory agents (NSAIDs), other muscarinic receptor antagonistst (i.e., antichlolinergic agents), antiinfective agents (e.g. antibiotics or antivirals) and antihistamines. The other therapeutic agents can be used in the form of pharmaceutically acceptable salts or solvates. Additionally, if appropriate, the other therapeutic agents can be used as optically pure stereoisomers.

The following formulations illustrate representative pharmaceutical compositions of the present invention:

### FORMULATION EXAMPLE A

Hard gelatin capsules for oral administration are prepared as follows:

| Ingredients | Amount |
|---|---|
| Salt of the Invention | 100 mg |
| Lactose (spray-dried) | 200 mg |
| Magnesium stearate | 10 mg |

Representative Procedure: The ingredients are throughly blended and then loaded into a hard gelatine capsule (310 mg of composition per capsule).

### FORMULATION EXAMPLE B

Hard gelatin capsules for oral administration are prepared as follows:

| Ingredients | Amount |
|---|---|
| Salt of the Invention | 20 mg |
| Starch | 89 mg |
| Microcrystalline cellulose | 89 mg |
| Magnesium stearate | 2 mg |

Representative Procedure: The ingredients are throughly blended and then passed through a No. 45 mesh U.S. sieve and loaded into a hard gelatin capsule (200 mg of composition per capsule).

### FORMULATION EXAMPLE C

Capsules for oral administration are prepared as follows:

| Ingredients | Amount |
|---|---|
| Salt of the Invention | 100 mg |
| Polyoxyethylene sorbitan monooleate | 50 mg |
| Starch powder | 250 mg |

Representative Procedure: The ingredients are throughly blended and then loaded into a gelatin capsule (300 mg of composition per capsule).

### FORMULATION EXAMPLE D

Tablets for oral administration are prepared as follows:

| Ingredients | Amount |
|---|---|
| Salt of the Invention | 10 mg |
| Starch | 45 mg |
| Microcrystalline cellulose | 35 mg |
| Polyvinylpyrrolidone (10 wt. % in water) | 4 mg |
| Sodium carboxymethyl starch | 4.5 mg |
| Magnesium stearate | 0.5 mg |
| Talc | 1 mg |

Representative Procedure: The active ingredient, starch and cellulose are passed through a No. 45 mesh U.S. sieve and mixed throughly. The solution of polyvinylpyrrolidone is mixed with the resulting powders, and this mixture is then passed through a No. 14 mesh U.S. sieve. The granules so produced are dried at 50-60° C. and passed through a No. 18 mesh U.S. sieve. The sodium carboxymethyl starch, magnesium stearate and talc (previously passed through a No. 60 mesh U.S. sieve) are then added to the granules. After mixing, the mixture is compressed on a tablet machine to afford a tablet weighing 100 mg.

### FORMULATION EXAMPLE E

Tablets for oral administration are prepared as follows:

| Ingredients | Amount |
|---|---|
| Salt of the Invention | 100 mg |
| Microcrystalline cellulose | 400 mg |
| Silicon dioxide fumed | 10 mg |
| Stearic acid | 5 mg |

Representative Procedure: The ingredients are throughly blended and then compressed to form tablets (515 mg of composition per tablet).

### FORMULATION EXAMPLE F

Single-scored tablets for oral administration are prepared as follows:

| Ingredients | Amount |
|---|---|
| Salt of the Invention | 100 mg |
| Cornstarch | 50 mg |
| Croscarmellose sodium | 25 mg |
| Lactose | 120 mg |
| Magnesium stearate | 5 mg |

Representative Procedure: The ingredients are throughly blended and compressed to form a single-scored tablet (200 mg of compositions per tablet).

### FORMULATION EXAMPLE G

A suspension for oral administration is prepared as follows:

| Ingredients | Amount |
|---|---|
| Salt of the Invention | 1.0 g |
| Fumaric acid | 0.5 g |
| Sodium chloride | 2.0 g |
| Methyl paraben | 0.15 g |
| Propyl paraben | 0.05 g |
| Granulated sugar | 25.5 g |
| Sorbitol (70% solution) | 12.85 g |
| Veegum k (Vanderbilt Co.) | 1.0 g |
| Flavoring | 0.035 mL |
| Colorings | 0.5 mg |
| Distilled water | q.s. to |
| | 100 mL |

Representative Procedure: The ingredients are mixed to form a suspension containing 100 mg of active ingredient per 10 mL of suspension.

### FORMULATION EXAMPLE H

A dry powder for administration by inhalation is prepared as follows:

| Ingredients | Amount |
|---|---|
| Salt of the Invention | 1.0 mg |
| Lactose | 25 mg |

Representative Procedure: The active ingredient is micronized and then blended with lactose. This blended mixture is then loaded into a gelatin inhalation cartridge. The contents of the cartridge are administered using a powder inhaler.

### FORMULATION EXAMPLE I

A dry powder for administration by inhalation in a metered dose inhaler is prepared as follows:

Representative Procedure: A suspension containing 5 wt. % of a salt of the invention and 0.1 wt. % lecithin is prepared by dispersing 10 g of active compound as micronized particles with mean size less than 10 μm in a solution formed from 0.2 g of lecithin dissolved in 200 mL of demineralized water. The suspension is spray dried and the resulting material is micronized to particles having a mean diameter less than 1.5 μm. The particles are loaded into cartridges with pressurized 1,1,1,2-tetrafluoroethane.

### FORMULATION EXAMPLE J

An injectable formulation is prepared as follows:

| Ingredients | Amount |
|---|---|
| Salt of the Invention | 0.2 g |
| Sodium acetate buffer solution (0.4 M) | 2.0 mL |
| HCl (0.5 N) or NaOH (0.5 N) | q.s. to pH 4 |
| Water (distilled, sterile) | q.s. to |
| | 20 mL |

Representative Procedure: The above ingredients are blended and the pH is adjusted to 4±0.5 using 0.5 N HCl or 0.5 N NaOH.

### FORMULATION EXAMPLE K

Capsules for oral administration are prepared as follows:

| Ingredients | Amount |
|---|---|
| Salt of the Invention | 40.05 mg |
| Microcrystalline cellulose (Avicel PH 103) | 259.2 mg |
| Magnesium stearate | 0.75 mg |

Representative Procedure: The ingredients are throughly blended and then loaded into a gelatin capsule (Size #1, White, Opaque) (300 mg of composition per capsule).

### FORMULATION EXAMPLE L

Capsules for oral administration are prepared as follows:

| Ingredients | Amount |
|---|---|
| Salt of the Invention | 99.2 mg |
| Microcrystalline cellulose (Avicel PH 103) | 100.05 mg |
| Magnesium stearate | 0.75 mg |

Representative Procedure: The ingredients are throughly blended and then loaded into a gelatin capsule (Size #1, White, Opaque) (200 mg of composition per capsule).

Utility

The 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention are useful as muscarinic receptor antagonists and therefore, such salts are expected to be useful for treating medical conditions mediated by muscarinic receptors, i.e., any medical condition that is ameliorated by treatment with a muscarinic receptor antagonist. Such medical conditions include, by way of example, genitourinary tract disorders, such as overactive bladder or detrusor hyperactivity and their symptoms; gastrointestinal tract disorders, such as irritable bowel syndrome, diverticular disease, achalasia, gastrointestinal hypermotility disorders and diarrhea; respiratory tract disorders, such as chronic obstructive pulmonary disease, asthma and pulmonary fibrosis; cardiac arrhythmias, such as sinus bradycardia; Parkinson's disease; cognitive disorders, such as Alzheimer's disease; dismenorrhea; and the like.

In particular, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention are useful for treating smooth muscle disorders in mammals, including humans. Such smooth muscle disorders include, by way of illustration, overactive bladder, asthma, chronic obstructive pulmonary disease and irritable bowel syndrome.

When used to treat smooth muscle disorders or other conditions mediated by muscarinic receptors, the compounds of this invention will typically be administered orally, rectally, parenterally or by inhalation in a single daily dose or in multiple doses per day. The amount of active agent administered per dose or the total amount administered per day will typically be determined by the patient's physician and will depend on such factors as the nature and severity of the patients condition, the condition being treated, the age and general health of the patient, the tolerance of the patient to the active agent, the route of administration and the like.

Typically, suitable doses for treating smooth muscle disorders or other disorders mediated by muscarinic receptors will range from about 0.01 to about 50 mg/kg/day of active agent; including from about 0.02 to about 10 mg/kg/day; such as 0.1 to 1 mg/kg/day. For an average 70 kg human, this would amount to about 0.7 to about 3500 mg per day of active agent, including 7 to 70 mg per day.

In a one embodiment, the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine naphthalene-1,5-disulfonic acid salts of this invention are used to treat overactive bladder. When used to treat overactive bladder, the salts of this invention will typically be administered orally in a single daily dose or in multiple doses per day; preferably in a single daily dose. Preferably, the dose for treating overactive bladder will range from about 1 to about 200 mg/day; such as 5 to 100 mg/day.

In another embodiment, the salts of this invention are used to treat a respiratory disorder, such as chronic obstructive pulmonary disease or asthma. When used to treat chronic obstructive pulmonary disease or asthma, the salts of this invention will typically be administered by inhalation in a single daily dose or in multiple doses per day. Preferably, the dose for treating chronic obstructive pulmonary disease or asthma will range from about 10 μg/day to about 10 mg/day.

In yet another embodiment, the salts of this invention are used to treat irritable bowel syndrome. When used to treat irritable bowel syndrome, the salts of this invention will typically be administered orally or rectally in a single daily dose or in multiple doses per day. Preferably, the dose for treating irritable bowel syndrome will range from about 1.0 to about 2000 mg/day.

If desired, the salts of this invention can be administered in combination with other therapeutic agents, such as those listed in the commonly-assigned U.S. patent application disclosed in the Background section of this application.

Among other properties, the compound of formula I and salts thereof have been found to be potent inhibitors of M₂ muscarinic receptor activity. Numerous in vitro and in vivo assays for demonstrating muscarinic receptor activity are well-known to those skilled in the art. For example, representative assays are described in further detail in the Examples below; and in the commonly-assigned U.S. patent application disclosed in the Background section of this application.

### EXAMPLES

The following synthetic and biological examples are offered to illustrate this invention and are not to be construed in any way as limiting the scope of this invention. In the examples below, the following abbreviations have the following meanings unless otherwise indicated. Abbreviations not defined below have their generally accepted meaning.

| | |
|---|---|
| BSA = | bovine serum albumin |
| CHO = | Chinese hampster ovary |
| DCM = | dichloromethane |
| DIPEA = | diisopropylethylamine |
| DME = | ethylene glycol dimethyl ether |
| DMSO = | dimethyl sulfoxide |
| dPBS = | Dulbecco's phosphate buffered saline, without |
| | CaCl₂ and MgCl₂ |
| EDTA = | ethylenediaminetetraacetic acid |
| EtOAc = | ethyl acetate |
| FBS = | fetal bovine serum |
| FTIR = | Fourier transform infrared |
| HEPES = | 4-(2-hydroxyethyl)-1-piperazine-ethanesulfonic acid |
| hM_{1 =} | human muscarinic receptor subtype 1 |
| hM_{2 =} | human muscarinic receptor subtype 2 |
| hM_{3 =} | human muscarinic receptor subtype 3 |
| hM_{4 =} | human muscarinic receptor subtype 4 |
| hM_{5 =} | human muscarinic receptor subtype 5 |
| HPLC = | high performance liquid chromatography |
| K_{i =} | inhibition dissociation constant |
| MS = | mass spectrometry |
| MTBE = | methyl tert-butyl ether |
| [³H]NMS = | l-[N-methyl-³H]scopolamine methyl chloride |
| TEA = | triethylamine |
| THF = | tetrahydrofuran |
| TLC = | thin layer chromatography |
| TFA = | trifluoroacetic acid |
| VIBC = | volume-induced bladder contraction |
| VIBC_{Amp =} | volume-induced bladder contraction amplitude |

All temperatures reported in the following examples are in degrees Celsius (° C.) unless otherwise indicated. Also, unless noted otherwise, reagents, starting materials and solvents were purchased from commercial suppliers (such as Aldrich, Fluka, Sigma and the like) and were used without further purification.

HPLC was conducted using an Agilient 1100 HPLC or equivalent instrument under the following conditions as indicated:

| | |
|---|---|
| HPLC Method A: | |
| Column: | Agilent Zorbax ® Bonus-RP 5μ 4.6 × 250 mm |
| Detector Wavelength: | 214 nm |
| Column Temperature: | 40° C. |
| Flow Rate: | 1.0 mL/min |
| Mobile Phases: | A = 2% acetonitrile, 98% water, 0.1% TFA |
| | B = 90% acetonitrile, 10% water, 0.1% TFA |
| Injection Volume: | 5 μL |
| Run Time: | 62 min |
| Gradient: | 2-40% B in A |
| HPLC Method B: | |
| Column: | YMC ODSA 5μ C18 4.6 × 50 mm |
| Detector Wavelength: | 220 nm |
| Column Temperature: | 35° C. |
| Flow Rate: | 4.0 mL/min |
| Mobile Phases: | A = 10% methanol, 90% water, 0.1% TFA |
| | B = 90% methanol, 10% water, 0.1% TFA |
| Injection Volume: | 5 μL |
| Run Time: | 5 min |
| Gradient: | 0-100% B in A |
| HPLC Method C: | |
| Column: | Inertsil ODS-2 C18 |
| Detector Wavelength: | 254 nm |
| Column Temperature: | 35° C. |
| Flow Rate: | 1.0 mL/min |
| Mobile Phases: | A = 5% methanol, 95% water, 0.1% TFA |
| | B = 95% methanol, 5% water, 0.1% TFA |
| Injection Volume: | 5 μL |
| Run Time: | 15 min |
| Gradient: | 0-100% B in A |
| HPLC Method D: | |
| Column: | ACE 5 C18, 4.6 mm × 25 cm |
| Detector: | DAD1, Signal = 230 nm/10 nm, Ref = 360 nm |
| Column Temperature: | 45° C. |
| Flow Rate: | 1.5 mL/min |
| Mobile Phases: | A = 20 mM TEA (pH 5.65)/acetonitrile |
| | (98:2; v/v) |
| | B = 100 mM TEA (pH 5.5)/acetonitrile |
| | (20:80; v/v) |
| Injection Volume: | 20 μL |
| Run Time: | 38 min |
| Gradient: | 10-80% B in A |

### Example 1

### Preparation of (S)-3-(1-Carbamoyl-1,1-diphenylmethyl)pyrrolidine

### Step A—Preparation of (S)-1-Benzyl-3-(p-toluenesulfonyloxy)pyrrolidine

To a stirred solution of (S)-1-benzyl-3-pyrrolidinol (44.3 g, 0.25 mol) and 1,4-diazabicyclo[2.2.2]octane (33.7 g, 0.3 mol) in 250 mL of tert-butyl methyl ether under an atmosphere of nitrogen at 0° C., was added p-toluenesulfonyl chloride (52.4 g, 0.275 mol) portion-wise over 20 min. The reaction mixture was stirred at 0° C. for 1 h. The ice bath was removed and the mixture was stirred at ambient temperature overnight (20±5 h). Ethyl acetate (100 mL) was added, followed by saturated aqueous sodium bicarbonate solution (250 mL). The resulting mixture was stirred at ambient temperature for 1 h. The layers were separated and the organic layer was washed with saturated aqueous sodium bicarbonate solution (250 mL); saturated aqueous ammonium chloride solution (250 mL); saturated aqueous sodium chloride solution (250 mL); and then dried over sodium sulfate (80 g). The sodium sulfate was filtered off and washed with ethyl acetate (20 mL) and the solvent was removed in vacuo to give 78.2 g of the title intermediate as an off-white solid (94% yield; 95% purity by HPLC Method B).

### Step B—Preparation of (S)-1-Benzyl-3-(1-cyano-1,1-diphenylmethyl)pyrrolidine

To a stirred solution of diphenylacetonitrile (12.18 g, 61.8 mmol) in anhydrous THF (120 mL) at 0° C., potassium tert-butoxide (10.60 g, 94.6 mmol) was added over 5 min. The reaction mixture was stirred at 0° C. for 1 h. To the reaction mixture at 0° C. was added (S)-1-benzyl-3-(p-toluenesulfonyloxy)-pyrrolidine (20.48 g, 61.3 mmol) in one portion. The cold bath was removed and the reaction mixture was stirred for 5-10 min at which time the reaction mixture had become a brown homogeneous solution. The reaction mixture was then heated at 40° C. overnight (20±5 h). The reaction mixture (bright yellow suspension) was allowed to cool to room temperature before adding water (150 mL). Most of the THF was then removed in vacuo and isopropyl acetate (200 mL) was added. The layers were separated and the organic layer was washed with saturated aqueous ammonium chloride solution (150 mL); saturated aqueous sodium chloride solution (150 mL); and then dried over sodium sulfate (50 g). The sodium sulfate was filtered off and washed with isopropyl acetate (20 mL) and the solvent was removed in vacuo to give 23.88 g of the title intermediate as a light brown oil (>99% yield, 75% purity by HPLC Method B, contaminated mainly with excess diphenylacetonitrile).

### Step C—Preparation of (S)-3-(1-Cyano-1,1-diphenylmethyl)pyrrolidine

(S)-1-Benzyl-3-(1-cyano-1,1-diphenylmethyl)pyrrolidine was dissolved in isopropyl acetate (ca.1 g/10 mL) and the solution was mixed with an equal volume of 1N aqueous hydrochloric acid. The resulting layers were separated and the aqueous layer was extracted with an equal volume of isopropyl acetate. The organic layers were combined, dried over sodium sulfate and filtered. The solvent was removed in vacuo to afford (S)-1-benzyl-3-(1-cyano-1,1-diphenylmethyl)pyrrolidine hydrochloride as a light yellow foamy solid. (Note: This hydrochloride salt can also be prepared during the work-up of Step B).

To a stirred solution of (S)-1-benzyl-3-(1-cyano-1,1-diphenylmethyl)pyrrolidine hydrochloride (8.55 g, 21.98 mmol) in methanol (44 mL) was added palladium on carbon (1.71 g) and ammonium formate (6.93 g, 109.9 mmol). The reaction mixture was heated to 50° C. with stirring for 3 h. The reaction was cooled to ambient temperature and water (20 mL) was added. The resulting mixture was filtered through a pad of Celite, washing with methanol (20 mL). The filtrate was collected and most of the methanol was removed in vacuo. The residue was mixed with isopropyl acetate (100 mL) and 10% aqueous sodium carbonate (50 mL). The resulting layers were separated and the aqueous layer was extracted with isopropyl acetate (50 mL). The organic layers were combined and dried over sodium sulfate (20 g). The sodium sulfate was filtered off and washed with isopropyl acetate (20 mL). The solvent was removed in vacuo to afford 5.75 g of the title intermediate as a light yellow oil (99.7% yield, 71% purity by HPLC).

### Step D—Preparation of (S)-3-(1-Carbamoyl-1,1-diphenylmethyl)pyrrolidine

A 200 mL flask with a magnetic stir bar and a nitrogen inlet was charged with (S)-3-(1-cyano-1,1-diphenylmethyl)pyrrolidine (2.51 g) and 80% H₂SO₄ (19.2 mL; pre-prepared with 16 mL of 96% H₂SO₄ and 3.2 mL of H₂O). The reaction mixture was then heated at 90° C. for 24 h or until starting material was consumed as indicated by HPLC. The reaction mixture was allowed to cool to room temperature and then poured onto ice (ca. 50 mL by volume). A 50% aqueous sodium hydroxide solution was added slowly to the mixture with stirring over an ice bath until the pH was about 12. Dichloromethane (200 mL) was added and mixed with the aqueous solution at which time sodium sulfate precipitated out and was filtered off. The filtrate was collected and the layers were separated. The aqueous layer was extracted with dichloromethane (100 mL) and the organic layers were combined and dried with over sodium sulfate (5 g). The sodium sulfate was filtered off and washed with dichloromethane (10 mL). The solvent was removed in vacuo to give the crude product as a light yellow foamy solid (ca. 2.2 g, 86% purity by HPLC).

The crude product was dissolved in ethanol (18 mL) with stirring. To this solution was added a warm solution of L-tartaric acid (1.8 g) in ethanol (14 mL) and the resulting mixture was stirred overnight (15±5 h). The resulting precipitate was isolated by filtration to give an off-white solid (ca. 3.2 g, >95% purity by HPLC). Methanol (15 mL) was added to this solid and the resulting slurry was stirred at 70° C. overnight (15 h). The slurry was allowed to cool to ambient temperature and a white solid (˜2.6 g, >99% purity by HPLC) was obtained after filtration. To this solid was added ethyl acetate (30 mL) and 1 N aqueous sodium hydroxide (25 mL). This mixture was mixed until two distinct layers formed and then the layers were separated and the aqueous layer was extracted with ethyl acetate (20 mL). The organic layers were combined and dried over sodium sulfate (10 g). The sodium sulfate was removed by filtration and the solvent was evaporated in vacuo to afford 1.55 g of the title intermediate as an off-white foamy solid (58% yield; >99% purity by HPLC Method C).

### Example 2

### Preparation of 4-Methoxypyridine-3-carboxaldehyde

tert-Butyllithium (90.6 mL, 154 mmol; 1.7 M in pentane) was added via cannula to a stirred solution of tetrahydrofuran (380 mL) under an atmosphere of nitrogen at room temperature. The reaction mixture was cooled to −78° C. before adding 2-bromomesitylene (11.3 mL, 74.1 mmol) dropwise. The reaction mixture was allowed to stir for 1 hour at −78° C. To the reaction mixture at −78° C. was added 4-methoxypyridine (5.79 mL, 57 mmol) dropwise, and the resulting mixture was stirred at −23° C. for 3 hours. The reaction mixture was then re-cooled to −78° C. and dimethylformamide (6.62 mL, 85.5 mmol) was added and stirring was continued for 1 hour at −78° C. The reaction mixture was quenched slowly at −78° C. with saturated aqueous sodium chloride solution (100 mL) and allowed to warm to room temperature slowly. To the reaction mixture was added diethyl ether (200 mL) and the layers were separated. The aqueous layer was extracted with diethyl ether (2×150 mL) and the combined organic layers were dried over potassium carbonate (20 g). The potassium carbonate was removed by filtration and washed with diethyl ether (100 mL) and the solvent removed under reduced pressure. The resulting crude 4-methoxy-3-pyridinecarboxaldehyde was purified by column chromatography (SiO₂, 5:95 ethanol:ethyl acetate) to give 4.79 g of the title intermediate as a yellow solid (61% yield; >98% purity by ¹H NMR).

Analytical Data: ¹H NMR (300 MHz, CDCl₃) δ 10.43 (s, 1H, CHO), 8.87 (s, 1H, ArH), 8.63 (d, 1H, J=6, ArH), 6.92 (d, 1H, J=6, ArH), 3.98 (s, 3H, CH₃O).

### Example 3A

### Preparation of 4-Isopropylamino-1-(4-methoxypyrid-3-ylmethyl)piperidine

### Monobenzoic Acid Salt

### Step A—Preparation of 1-Benzyl-4-isopropylaminopiperidine

A solution of 4-amino-1-benzylpiperidine (45.8 g, 0.24 mol) and acetone (531 mL) was stirred at room temperature for 12 hours. The reaction mixture was then reduced to ca.150 mL in vacuo. To this mixture was added methanol (100 mL) and the resulting mixture was cooled to 5° C. in an ice/water bath. Sodium triacetoxyborohydride (61.2 g, 0.29 mol) in methanol (350 mL), previously cooled to 5° C. in an ice/water bath, was added and this reaction mixture was stirred at 5° C. for 0.5 hours. The ice/water bath was removed and the reaction mixture was stirred for 2 hours at room temperature and then re-cooled to 5° C. in ice/water bath. To this mixture was added concentrated hydrochloric acid (75 mL) until the pH of the reaction mixture was about 3. This mixture was stirred for 1 hour and then concentrated in vacuo to about 600 mL and 1 N aqueous hydrochloric acid (200 mL) was added to dissolve the solids. The aqueous layer was washed with isopropyl acetate (400 mL) and the layers were separated. The aqueous layer was adjusted to pH 12 with 10 N aqueous sodium hydroxide (200 mL) and isopropylacetate (600 mL) was added. This mixture was stirred for 1 hour at room temperature and then the layers were separated and the organic layer washed with saturated aqueous sodium chloride solution (600 mL) and dried over sodium sulfate (80 g). The sodium sulfate was filtered off and washed with ethyl acetate (20 mL). The solvent was removed in vacuo to give 52.0 g of the title intermediate as a yellow oil (95% yield).

### Step B—Preparation of 1-Benzyl-4-(N-tert-butoxycarbonyl-N-isopropylamino)piperidine

A solution of 1-benzyl-4-isopropylaminopiperidine (69.7 g, 0.30 mol) in dichloromethane (200 mL) was cooled to 5° C. in an ice/water bath. To this solution was added di-tert-butyl dicarbonate (72.0 g, 0.33 mol) in dichloromethane (180 mL). The temperature did not rise more than 5° C. during the addition. The reaction mixture was stirred at 5° C. for 0.5 hour and then the ice/water bath was removed. The reaction mixture was stirred for 24 hours and was then concentrated in vacuo. The resulting yellow oil was placed under vacuum for 2 hours at which time it slowly crystallized to afford 98 g of the title intermediate as light yellow needle-shaped crystals (>99% yield).

### Step C—Preparation of 4-(N-tert-Butoxycarbonyl-N-isopropylamino)piperidine

A solution of 1-benzyl-4-(N-tert-butoxycarbonyl-N-isopropylamino)piperidine (79.0 g, 0.24 mol) in ethanol (140 mL) was flushed with nitrogen for 15 minutes. This solution was then added to a 2 L Parr flask containing a mixture of 10% palladium on carbon (15.8 g; ca.50% wt. water) in ethanol (100 mL), which solution had been flushed with nitrogen for 15 minutes. This reaction mixture was placed on a Parr Shaker under hydrogen at 50 psi for 24 hours. The reaction mixture was filtered through a pad of Celite and the Celite washed with ethanol. The filtrate was then concentrated in vacuo to afford 57.0 g of the title intermediate as a white solid (>99% yield).

### Step D—Preparation of 4-(N-tert-Butoxycarbonyl-N-isopropylamino)-1-(4-methoxypyrid-3-ylmethyl)piperidine

A solution of 4-(N-tert-butoxycarbonyl-N-isopropylamino)piperidine (118 g, 0.49 mol) in dichloroethane (600 mL) was stirred at room temperature for 1 hour, and then 4-methoxypyridine-3-carboxylate (63.5 g, 0.46 mol) was added. The resulting solution was stirred at room temperature for 2.5 hours and then cooled to 5° C. in an ice/water bath. Sodium triacetoxyborohydride (124 g, 0.58 mol) in dichloroethane (600 mL) was added and the reaction mixture was stirred at 5° C. for 15 minutes. The ice bath was then removed and reaction mixture was stirred for 4 hours at room temperature. Acetic acid (30 mL) was then added to the reaction mixture and the resulting mixture was stirred for 0.5 hours, and then concentrated to half its original volume. This solution was cooled in a dry ice/acetone bath and 10 N aqueous sodium hydroxide (350 mL) was added. This mixture was stirred for 0.5 hours and then the organic layer was separated and washed with 1 N aqueous sodium hydroxide (400 mL). The aqueous layer was then washed three times with dichloromethane (400 mL) and the combined organic layers were dried over sodium sulfate (40 g). The sodium sulfate was filtered off and washed with dichloromethane (100 mL) and the combined organic layers were concentrated in vacuo to give 177 g of the title intermediate as a yellow oil (>99% yield; 74% purity by GC).

### Step E—Preparation of 4-Isopropylamino-1-(4-methoxypyrid-3-ylmethyl)piperidine

A solution of 4-(N-tert-butoxycarbonyl-N-isopropylamino)-1-(4-methoxypyrid-3-ylmethyl)piperidine (17.0 g, 0.047 mol) in dioxane (93 mL) was cooled to 5° C. in ice/water bath. To this solution was added concentrated hydrochloric acid (40 mL) and the resulting mixture was stirred 5° C. for 15 minutes. The ice/water bath was then removed and the reaction mixture was stirred for 12 hours. The reaction mixture was then concentrated in vacuo to dryness, diluted with dichloromethane (100 mL) and 10 N aqueous sodium hydroxide was added slowly (CAUTION: very exothermic) until the pH was 14. The mixture was stirred for 0.5 hours and the organic layer was then separated and the aqueous layer was washed three times with dichloromethane (200 mL). The organic layers were then separated and dried over sodium sulfate (10 g). The sodium sulfate was removed by filtration and the organic layer was concentrated in vacuo to give 7.8 g of the title intermediate as a yellow oil (65% yield; 83% purity by GC).

### Step F—Preparation of 4-Isopropylamino-1-(4-methoxypyrid-3-ylmethyl)piperidine Monobenzoic Acid Salt

To a 1L reaction flask equipped with a mechanical stirrer and a nitrogen inlet was added 4-isopropylamino-1-(4-methoxypyrid-3-ylmethyl)piperidine (45.7 g, 0.174 mol) and 200 mL of MTBE. The resulting mixture was heated to 50-55° C. to dissolve the solid. To this solution was added a solution of benzoic acid (21.3 g, 0.174 mol) in 100 mL of MTBE at 50-55° C. (Note: Heat may be needed to dissolve the benzoic acid in MTBE). This mixture was stirred at 50-55° C. for 30 minutes and then stirred at room temperature for 16 hours. The resulting solid was filtered and washed with 50 mL of MTBE and then dried under vacuum at 40° C. for 16 hours to give 54.9 g of the title intermediate as a white solid (82% yield; ≧99% purity).

### Example 3B

### Preparation of 4-Isopropylamino-1-(4-methoxypyrid-3-ylmethyl)piperidine

### Monobenzoic Acid Salt

### Step A—Preparation of 1-Benzyl-4-isopropylaminopiperidine

To a 50 L 3-neck round-bottom reaction flask equipped with a mechanical stirrer, temperature probe, nitrogen inlet and cooling bath was added 4-amino-1-benzylpiperidine (2,000 g, 10.5 mol) and dichloromethane (20 L). Acetone (610.5 g, 10.5 mol) was added and the reaction mixture was stirred at room temperature for 2.5 hours. The reaction mixture was then cooled to 0° C. to 5° C. with an ice/methanol bath and sodium triacetoxyborohydride (2,673 g, 12.6 mol) was added while maintaining the temperature of the reaction mixture below 25° C. The cooling bath was then removed and the reaction mixture was stirred until less than 1% starting material was present by GC analysis (about 3 hours). Concentrated hydrochloric acid was added until the pH of the reaction mixture was 7 (about 500 mL). The resulting slurry was filtered through a polypropylene filter pad and the solids were washed with dichloromethane (2×2 L). The solids were saved for use after concentration of the filtrate. The filtrate was concentrated at 40° C. until no condensate remained. In a 40 L separatory funnel, the solids and distillation residue were dissolved in water (15 L) and concentrated hydrochloric acid was added until the pH of the solution was 3 (about 2.5 L). The aqueous layer was then washed with dichloromethane (2×2 L). The pH of the aqueous layer was adjusted to 11 to 12 with 50% aqueous sodium hydroxide solution (about 4.5 L) and this mixture was extracted with dichloromethane (5×3L). The organic layers were combined, decolorized with charcoal (50 g) and dried over anhydrous magnesium sulfate (200 g). The solids were filtered off using a glass fiber filter pad and the filtrate was concentrated until no condensate remained to afford the title compound (2,336 g, 96% yield).

### Step B—Preparation of 4-Isopropylaminopiperidine

The product from Step A (18 g, 77 mmol) and methanol (200 mL) were added to a 500 mL round-bottom flask and the resulting mixture was stirred until a clear solution was obtained. Palladium on carbon (400 mg, 10%) in methanol (2 mL) was then added and the reaction mixture was placed under a hydrogen-filled balloon and stirred at ambient temperature for 18 hours. The reaction mixture was then filtered through a Celite pad to remove the catalyst and the filtrate was concentrated on a rotary evaporator to afford the title compound as a yellow-colored oil (11 g, quantitative yield).

### Step C—Preparation of 4-Isopropylamino-1-(4-methoxypyrid-3-ylmethyl)piperidine

4-Isopropylaminopiperidine (1.32 g, 9.3 mmol) and dichloromethane (40 mL) were added to a 100 mL round-bottom flask equipped with a cooling bath. 4-Methoxypyridine-3-carboxaldehyde (1.44 g, 10.5 mmol) was added and the reaction mixture was stirred at room temperature for 1 hour. The reaction mixture was then cooled to 0° C. to 5° C. using a methanol/ice bath and sodium triacetoxyborohydride (2.54 g, 12 mmol) was added at such a rate so as to maintain the temperature of the reaction mixture less than 10° C. When the addition was complete, the reaction mixture was stirred at ambient temperature until less than 1% starting material was present by GC analysis (about 3 hours). Aqueous IN hydrochloric acid (20 mL) was then added and the layers were separated. The pH of aqueous layer was adjusted to 12 with aqueous 50% sodium hydroxide solution and the resulting mixture was stirred for 1 hour. The aqueous layer was then extracted with ethyl acetate (2×20 L) and the combined organic layers were decolorized with charcoal (1 g) and dried over anhydrous magnesium sulfate (5 g). The solids were removed by filtration through a glass fiber filter pad and the filtrate was concentrated under vacuum. The residue was further dried under high vacuum for 1 hour to give the title compound (2.1 g, 80% yield).

### Step D—Preparation of 4-Isopropylamino-1-(4-methoxypyrid-3-ylmethyl)piperidine Monobenzoic Acid Salt

Benzoic acid (1451 g, 11.9 mol) and MTBE (5.8 L) were added to a 50 L 3-necked round-bottom flask equipped with a mechanical stirrer, thermometer, nitrogen inlet and heating mantle. The resulting slurry was heated at 45° C. to 50° C. to dissolve the benzoic acid. A solution of 4-isopropylamino-1-(4-methoxypyrid-3-ylmethyl)piperidine (3130 g, 11.9 mol) in MTBE (13.7 L) was added at 45° C. to 50° C. and the resulting mixture was stirred at reflux (50° C. to 55° C.) for 30 minutes and then at ambient temperature for 16 hours. The reaction mixture was then cooled to 0° C. to 5° C. with an ice/methanol bath and stirred for 30 minutes at which time a solid had formed. The solid was filtered through a polypropylene filter pad and washed with MTBE (3×2 L) and ethyl ether (3×2 L). The solid was then tray dried in a vacuum oven at room temperature until a constant weight was obtained to provide the title compound (3,805 g, 82% yield).

### Example 4

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Method A

### Step A—Preparation of (S)-3-(1-Carbamoyl-1,1-diphenylmethyl)-1-(7-hydroxyhept-1-yl)pyrrolidine

To a stirred solution of (S)-3-(1-carbamoyl-1,1-diphenylmethyl)pyrrolidine (40 g, 142.7 mmol) and triethylamine (59.6 mL, 428 mmol) in acetonitrile (1.1 L) at 40° C. under a nitrogen atmosphere was added 7-bromo-1-heptanol (24 mL, 146 mmol) in acetonitrile (100 mL) dropwise. The reaction mixture was heated to 50° C. for 9 hours. The reaction mixture was allowed to cool before removing the solvent under reduced pressure. The crude residue was dissolved in dichloromethane (500 mL) and the organic layer washed with saturated aqueous sodium bicarbonate (2×300 mL), followed by water (300 mL) and saturated aqueous sodium chloride (300 mL), and then dried over magnesium sulfate (10 g). The magnesium sulfate was filtered off and washed with dichloromethane (100 mL). The solvent was then removed in vacuo to give the crude product which was purified on a short column (SiO₂) by varying the eluant from 19:1:0.1 to 3:1:0.1 CH₂Cl₂/MeOH/NH₄OH to give 31.35 g of the title intermediate as a white solid (56% yield; >95% purity by HPLC Method A).

### Step B—Preparation of (S)-3-(1-Carbamoyl-1-diphenvlmethvl)-1-(7-oxohept-1-yl)pyrrolidine

To a stirred solution of (S)-3-(1-carbamoyl-1,1-diphenylmethyl)-1-(7-hydroxyhept-1-yl)pyrrolidine (31.00 g, 78.57 mmol); N,N-diisopropylethylamine (68.4 mL, 392.8 mmol); and methyl sulfoxide (60.7 mL, 785.7 mmol) in dichloromethane (780 mL) under an atmosphere of nitrogen at −15° C., was added sulfur trioxide pyridine complex (37.5 g, 235.71 mmol) portion-wise over a 40 min. period. The reaction mixture was maintained between −10° C. and −20° C. during the addition. The reaction was then stirred in this temperature range for 40±10 min. Deionized water (300 mL) was added and the mixture was stirred for 10 minutes. The organic layer was separated and washed with deionized water (200 mL), followed by saturated aqueous sodium chloride (200 mL) and the organic layer was then dried with magnesium sulfate (10 g). The magnesium sulfate was filtered off and washed with dichloromethane (50 mL) and the solvent was reduced in vacuo. The resultant syrup was washed with petroleum ether (2×200 mL) to remove the remaining pyridine and DMSO and the resulting white solid was dried in vacuo to give 33.02 g of the title intermediate (98% yield; >93% purity by chiral HPLC Method A).

### Step C—Preparation of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

To a 50 mL flask equipped with a nitrogen inlet was added (S)-3-(1-carbamoyl-1,1-diphenylmethyl)-1-(7-oxohept-1-yl)pyrrolidine (2.36 g, 6.0 mmol); 4-isopropylamino-1-(4-methoxypyrid-3-ylmethyl)piperidine (1.61 g, 6.1 mmol) and dichloromethane (12 mL). This mixture was stirred at room temperature for 1 hour and then sodium triacetoxyborohydride (1.65 g, 7.8 mmol) was added and stirring was continued at room temperature for 20 hours (at which time essentially all of the starting pyrrolidine compound had reacted as determined by HPLC). The reaction was then quenched by the addition of 6 N aqueous hydrochloric acid (12 mL) and the layers were separated. The aqueous layer was washed with dichloromethane (12 mL) and, after separation, isopropyl acetate (40 mL) was added to the aqueous layer. The aqueous layer was then made basic to pH 14 by adding 10 N aqueous sodium hydroxide solution (alternatively, conc. ammonium hydroxide may be used). The layers were separated and the organic layer was washed with saturated aqueous sodium chloride solution (40 mL); and dried over sodium sulfate (5 g). The sodium sulfate was filtered off, and solvent was removed in vacuo to give 2.4 g of crude product as a light yellow foamy solid (63% yield; R_{f}=0.4 with CH₂Cl₂/MeOH/NH₄OH=88:10:2). The crude product was further purified by SiO₂ chromatography (60 g, SiO₂, CH₂Cl₂/MeOH/NH₄OH=90:10:1 (300 mL) to 85:15:1 (300 mL)). The appropriate fractions were combined to give 0.98 g of the title compound as a white solid (26% yield; 98% purity by HPLC Method A).

### Example 5

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Method B

### Step A—Preparation of Hex-5-yn-1-al

To a stirred solution of 5-hexyn-1-ol (10.0 g, 0.10 mol) stirring in dichloromethane (1 L) under an atmosphere of nitrogen, was added DMSO (71 mL, 1.0 mol) followed by DIPEA (174 mL, 1.0 mol). The reaction mixture was cooled to −15° C. and sulfur trioxide pyridine complex (79.6 g, 0.5 mol) was added in 10 g portions over 60 mins. The reaction mixture was stirred at −15° C. for 1 hour before examining by TLC (30% EtOAc/Hexane) to observe for complete consumption of the starting material. To the reaction mixture was added 1 N aqueous hydrochloric acid (1 L), and the organic layer was separated and washed with 1 N aqueous hydrochloric acid (3×500 mL), saturated aqueous sodium bicarbonate (500 mL), brine (1 L), dried over magnesium sulfate and the solvent reduced in vacuo to afford the title intermediate (NOTE: Product is volatile, use cold water bath and remove when solvent evaporated).

### Step B—Preparation of (S)-3-(1-Carbamoyl-1,1-diphenylmethyl)-1-(hex-5-yn-1-yl)pyrrolidine

To a stirred solution of (S)-3-(1-carbamoyl-1,1-diphenylmethyl)pyrrolidine (64.4 g, 0.23 mol); sodium triacetoxyborohydride (50.9 g, 0.24 mol) and acetic acid (13 mL, 0.23 mol) in dichloromethane (511 mL) at room temperature, was added a solution of hex-5-yn-1-al (26.14 g, 0.27 mol) in dichloromethane (256 mL). The reaction mixture stirred at room temperature overnight (ca. 8 hours) and then the reaction mixture was quenched by addition of concentrated hydrochloric acid (30 mL) and stirring was continued for 1 hour at room temperature. The mixture was then diluted with water (750 mL) and made basic to pH 5 using 10 N sodium hydroxide (18 mL). The layers were separated and the organic layer was washed with 1 N sodium hydroxide (200 mL). The organic layer was dried over magnesium sulfate (10 g); filtered and then concentrated in vacuo to afford 67.6 g of the title intermediate as a yellow gummy solid (83% yield).

### Step C—Synthesis of 4-{N-[7-(3-(S)-1-Carbamoyl-1,1-diphenylmethyl)pyrrolidin-1-yl)hept-2-yn-1-yl]-N-(isopropyl)amino}-1-(4-methoxypyrid-3-ylmethyl)piperidine

To a stirred solution of (S)-3-(1-carbamoyl-1,1-diphenylmethyl)-1-(hex-5-yn-1-yl)pyrrolidine (17.8 g, 49.4 mmol), paraformaldehyde (1.93 g, 64.2 mmol) and 4-isopropylamino-1-(4-methoxypyrid-3-ylmethyl)piperidine (14.3 g, 54.3 mmol) in THF (247 mL) under nitrogen at 55° C., was added copper (I) chloride (0.978 g, 9.88 mmol). The reaction mixture was stirred at 55° C. for 5 hours and then the solvent was removed under reduced pressure. The crude residue was dissolved in dichloromethane (250 mL) and filtered through Celite, washing with dichloromethane (50 mL). The filterate was washed with 5 N sodium hydroxide (3×100 mL) and the dried over magnesium sulfate (10 g). The solvent was then removed in vacuo to provide 29.8 g of the title intermediate as a pale yellow solid (95% yield).

### Step D—Preparation of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

The alkyne intermediate from Step C (28.4 g, 47 mmol) and p-toluenesulfonhydrazide (87.5 g, 470 mmol) were dissolved in DME (700 mL) and brought to reflux (ca. 85° C.). A solution of sodium acetate (77.1 g, 940 mmol) in water (470 mL) was then added dropwise at the rate of about 20 mL/hour and the reaction mixture was continually refluxed for 18 hours. The reaction mixture was then allowed to cool to room temperature and 10 N sodium hydroxide was added to adjust the pH to 12. The organic layer was separated and the aqueous layer was extracted with ethyl acetate (2×400 mL). The combined organic layers were washed with 1 N sodium hydroxide (2×350 mL) and then extracted using 1 N hydrochloric acid (2×350 mL). The combined acidic aqueous extracts were made basic to pH 12 with 10 N sodium hydroxide and extracted with ethyl acetate (2×400 mL). The combined organic layers were washed with saturated aqueous sodium chloride solution (400 mL), and dried over magnesium sulfate (10 g). The magnesium sulfate was filtered off and washed with ethyl acetate (200 mL) and the solvent removed in vacuo to give the title compound.

### Example 6

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Method C

### Step A—Preparation of 7,7-Dimethoxyheptanal

Cycloheptene (20.0 g, 0.208 mol) was added to a three-neck round-bottom flask containing low water UV-grade methanol (0.5 M concentration). The reaction mixture was cooled to −78° C., and ozone was bubbled through for 45 minutes. The solution was purged with nitrogen in order to prevent over oxidation. p-Toluenesulfonic acid (3.96 g, 0.021 mol) was added, and the reaction mixture was slowly warmed to 0° C. (two hours total reaction time). The acid was neutralized by adding excess solid sodium bicarbonate (69.9 g, 0.832 mol) and after the mixture was stirred for 15 minutes, dimethyl sulfide (28.6 g, 0.46 mol) was added. After 16 h, the reaction mixture was concentrated by solvent removal on rotary evaporator. Water was added (10 mL/g) and the heterogeneous mixture was stirred for 30 minutes. The crude product was extracted with MTBE (2×20 mL/g) and the combined organic extracts were dried with sodium sulfate and concentrated under reduced pressure. The crude product was purified by vacuum distillation (observed b.p. 80-85° C., at a pressure of about 1.0 mm) to give 28.95 g of the title intermediate.

### Step B—Preparation of (S)-3-(1-Carbamoyl-1,1-diphenylmethyl)-1-(7,7-dimethoxyhept-1-yl)pyrrolidine

To a three-necked 500 mL flask equipped with a mechanical stirrer, a nitrogen inlet, cooling bath, and a thermometer was added (S)-3-(1-carbamoyl-1,1-diphenylmethyl)pyrrolidine (25 g, 0.089 mol) and dichloromethane (200 mL). This mixture was cooled to about 0° C. and 7,7-dimethoxyheptanal (18.6 g, 0.107 mol) was added slowly. During the addition, the reaction temperature was maintained at 5° C. or less. The resulting mixture was stirred at 0° C. to 5° C. for 1 hour and then sodium triacetoxyborohydride (24.6 g, 0.116 mol) was then added over a 30 minute period. During this addition, the reaction temperature was also maintained at 5° C. or less. The resulting mixture was then stirred at 0 to 5° C. for 6 hours. The reaction was then quenched by adding 5% aqueous potassium carbonate solution (200 mL) while keeping the reaction temperature less than about 20° C. and the resulting mixture was stirred for 1 hour at room temperature. The organic layer was separated and washed with brine (100 mL) and then dried with sodium sulfate (20 g). The organic layer was then concentrated under vacuum to a volume of about 100 mL and this mixture was purified by silica gel chromatography eluting with a gradient of 1 to 10% v/v methanol in dichloromethane. The fractions containing the desired product were combined and concentration under vacuum to afford 28 g of the title intermediate as an oil (72% yield).

Analytical Data: ¹HNMR(CDCl₃) δ : 7.44-7.15 (m, 10H); 5.88 (s, 2H); 4.33 (t, J=6.7 Hz, 1H);3.70-3.58 (m, 1H); 3.30 (s, 6H); 3.10-2.92 (m, 3H); 2.76-2.64 (m, 1H); 2.61-2.52 (m, 2H); 2.30 (m, 1H); 2.20 (m, 1H); 1.56 (m, 4H); 1.26 (m, 7H).

Alternatively, this intermediate was prepared as follows: To a three-necked 50L flask equipped with a mechanical stirrer, a nitrogen inlet, cooling bath and a thermometer was added (S)-3-(1-carbamoyl-1,1-diphenylmethyl)pyrrolidine (2.5 kg, 8.93 mol) and dichloromethane (20 L) and this mixture was stirred until the solid dissolved. The reaction mixture was then cooled to 0° C. and 7,7-dimethoxy-heptanal (1.71 kg, 9.82 mol) was added slowly while maintaining the reaction temperature below 5° C. This reaction mixture was stirred at 0° C. to 5° C. for 1 hr and then sodium triacetoxyborohydride (2.27 kg, 10.72 mol) was added in small portions over 30 minutes while maintaining the reaction temperature below 5° C. The reaction mixture was then stirred at room temperature for 6 hrs. An aqueous 5% potassium carbonate solution (20 L) was then added while maintaining the reaction temperature below 20° C. and the reaction mixture was then stirred for 1 hr at room temperature. The layers were then separated and the organic layer was washed with brine (10 L) and then dried over sodium sulfate (2 kg) for about 3 hrs. After separating the organic layer from the sodium sulfate, the organic layer was concentrated to about 10 L under reduced pressure. This mixture was then purified by silica gel chromatography (40 kg) using the following sequence of eluents: dichloromethane (100 L); 3% MeOH, 97% DCM, as needed; 5% MeOH, 95% DCM, as needed; and 10% MeOH, 90% DCM, as needed. The fractions containing the desired intermediate were then combined (R_{f}0.3; 10% MeOH/90% DCM) and concentrated at a temperature less than 30° C. to afford 3.3 kg of the title intermediate.

### Step C—Preparation of (S)-3-(1-Carbamoyl-1,1-diphenylmethyl)-1-(7-oxohept-1-yl)pyrrolidine

To a three-necked 500 mL flask equipped with a mechanical stirrer, a nitrogen inlet, cooling bath, and a thermometer was added (S)-3-(1-carbamoyl-1,1-diphenylmethyl)-1-(7,7-dimethoxyhept-1-yl)pyrrolidine (16 g, 0.036 mol) and acetonitrile (100 mL). This mixture was cooled to about 10° C. and 100 mL of 1N aqueous hydrochloric acid was added while maintaining the reaction temperature at 20° C. or less. The resulting mixture was stirred at 20±5° C. for 2 hours. The reaction mixture was then extracted with dichloromethane (1×200 mL and 2×100 mL). The combined organic layers were washed with brine (200 mL) and dried with sodium sulfate (40 g). The organic layer was then concentrated under vacuum at about 25° C. to a volume of about 200 mL. This solution, containing the title intermediate as the hydrochloride salt, was used directly in the next step without further purification.

Alternatively, this intermediate was prepared as follows: To a three-necked 50 L flask equipped with a mechanical stirrer, a nitrogen inlet, cooling bath and a thermometer was added the intermediate from Step B (3.3 kg, 7.25 mol) and acetonitrile (15 L). This mixture was cooled to less than 10° C. and an aqueous 1 N hydrochloric acid solution (15 L) was added while maintaining the reaction temperature less than 20° C. The reaction mixture was then stirred at room temperature for 2 hrs. Dichloromethane (20 L) was then added and this mixture was stirred for 30 minutes and then separated. The aqueous layer was extracted with dichloromethane (2×10 L) and the combined organic layers were washed with brine (20 L) and dried over sodium sulfate (4 kg) for at least 3 hours. After separating the organic layer from the sodium sulfate, the organic layer was concentrated to about 20 L under reduced pressure at a temperature less than 25° C. This solution, containing about 1.5 kg of the title intermediate as the hydrochloride salt, was used in subsequent reactions without further purification. Alternatively, if desired, the solution can be further concentrated and the resulting residue purified by conventional procedures.

### Step D—Preparation of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

To a three-necked 500 mL flask equipped with a mechanical stirrer, a nitrogen inlet, cooling bath, and a thermometer was added 4-isopropylamino-1-(4-methoxypyrid-3-ylmethyl)piperidine benzoate (14.1 g, 0.036 mol) and (S)-3-(1-carbamoyl-1,1-diphenylmethyl)-1-(7-oxohept-1-yl)pyrrolidine hydrochloride salt solution (200 mL) from Step C above. This mixture was stirred at room temperature for 1 hour and then cooled to 10° C. to 15° C. Sodium triacetoxyborohydride (9.3 g, 0.044 mol) was added portionwise over 30 minutes and the resulting mixture was stirred at room temperature for 15 to 20 hours. The reaction mixture was then cooled to 0° C. to 10° C. and the reaction quenched by adding 6 N aqueous hydrochloric acid (200 mL) while maintaining the reaction temperature at 25° C. or less. The aqueous layer was separated and washed with dichloromethane (3×100 mL) and then made basic to about pH 12 by adding concentrated aqueous ammonium hydroxide. The resulting mixture was extracted with dichloromethane (1×200 mL and 1×100 mL) and the combined organic layers were washed with water (100 mL) and then concentration under vacuum. The resulting residue was dissolved in MTBE (250 mL) and the MTBE solution was then washed with water (3×100 mL), brine (100 mL), dried over sodium sulfate (30 g) and filtered. The MTBE solution was then concentrated under vacuum to give 19 g of the title compound as an oil (81.5% yield; 94.9% purity by HPLC Method D).

The title compound (1 g) was purified by silica gel chromatography eluting with a gradient of 3% to 10% v/v methanol in dichloromethane containing 0.5% concentrated ammonium hydroxide. The fractions containing the title compound were combined and concentrated under vacuum to give 0.6 g to the title compound as an oil (98.6% purity by HPLC Method D).

Analytical Data: ¹HNMR(CDCl₃) δ: 8.41 (s, 1H); 8.39 (d, J=5.7 Hz, 1H); 7.44-7.41 (m, 2H); 7.33-7.14 (m, 8H); 6.76 (d, J=5.6 Hz, 1H); 5.74 (s, 2H); 3.85 (s, 3H); 3.52 (s, 2H); 3.42 (m 1H); 3.10-2.78 (m, 4H); 2.70-2.25 (m, 8H); 2.10-1.85 (m, 3H); 1.70-1.52 (m, 4H); 1.48-1.15 (m, 10H); 0.97 (d J=6.6 Hz, 6H).

### Example 7

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Naphalene-1,5-disulfonic Acid Salt

To a 100 mL flask was added 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine (10.45 g, 16.33 mmol) and methanol (53 mL). After the compound dissolved, the solution was cooled to about 10° C. and naphthalene-1,5-disulfonic acid tetrahydrate (4.37 g, 15.15 mmol) was added portionwise while maintaining the reaction temperature below 10° C. When the addition was complete, the reaction mixture was stirred for 30 minutes. The reaction mixture was then added slowly over 2 h to a mixture of isopropanol (530 mL) and MTBE (265 mL) at 0-5° C. This mixture was then stirred for 1 hour and the resulting solid was filtered and washed with MTBE (50 mL). The solid was then dried under vacuum at room temperature for 5 days. During this time, the solid was removed from the drying chamber on days 2 and 4 and run through a ball mill (400 rpm, 3×2 minutes). This process provided 12 g of the title salt (80% yield) as an amorphous white powder (98.9% purity by HPLC Method D; 65.1% free base content relative to reference standard).

Analytical Data: FTIR (cm⁻¹): 1671.7 (w), 1593.5 (w), 1497.6 (w), 1291.2 (w), 1220.9 (m), 1180.3 (m), 1030.1 (s); MS m/z 640.8 (MH⁺ free base); 928.8 (MH⁺ free base+salt); Anal. Calcd for C₅₀H₆₅N₅O₈S₂: C, 63.30; H, 7.52; N, 7.14; S, 6.15. Found C, 63.53; H, 7.65; N, 7.23; S, 6.30.

This salt had a molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine of about 0.95 to 1 as determined by ¹H NMR (ratio of naphthalene ring protons to pyridine ring protons).

If desired, the naphthalene-1,5-disulfonic acid salts of this invention can be further purified using the following slurry procedure: To the naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine (8.0 g) was added isopropanol (80 mL). The resulting slurry was stirred for 6 hrs at room temperature. The mixture was then filtered and the solids were washed with MTBE (2×40 mL) and then dried under vacuum and nitrogen for 16 hours to afford 7.8 g to the title compound (97.5% recovery by weight).

### Example 8

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Naphalene-1,5-disulfonic Acid Salt

### Step A—Preparation of (7,7-Dimethoxyheptyl)isopropyl-[1-(4-methoxypyridin-3-ylmethyl)piperidin-4-yl]amine

To a reactor containing dichloromethane (4 L) was added 4-isopropylamino-1-(4-methoxypyrid-3-ylmethyl)piperidine monobenzoic acid salt (1.5 kg, 3.89 mol) while maintaining the temperature of the mixture at −5° C. to 5° C. The container used to add the salt was rinsed with dichloromethane (1.5 L) and the rinse was added to the reaction mixture. The temperature of the reaction mixture was then adjusted to 0° C. to 5° C. and 7,7-dimethoxyheptanal (790 g, 4.25 mol, 93.8% purity by GC) was added while maintaining the temperature of the reaction mixture between 0° C. to 5° C. The container used to add the 7,7-dimethoxyheptanal was rinsed with dichloromethane (0.8 L) and the rinse was added to the reactor. The resulting reaction mixture was then stirred at 0° C. to 5° C. for 1 hour. Sodium triacetoxyborohydride (1.07 kg, 5.05 mol) was then added in 7 equal portions over a period of 1 hour while maintaining the temperature of the reaction mixture between −5° C. to 5° C. The container used to add the sodium triacetoxyborohydride was rinsed with dichloromethane (0.8 L) and the rinse was added to the reaction mixture. The reaction mixture was then stirred at 0° C. to 5° C. for 21 hours. An aqueous solution of potassium carbonate (500 g) in deionized water (8.6 L) was then added to the reaction mixture while maintaining the temperature of the mixture between to 0° C. to 25° C. The resulting mixture was stirred for 2 hours at a temperature between 15° C. to 25° C. The layers were then allowed to separate over a period of 30 minutes and the organic layer was collected. The washing procedure with aqueous potassium carbonate solution was repeated 2 times. To the organic layer was then added an aqueous solution of sodium chloride (5.7 kg) in deionized water (15 L) while maintaining the temperature between to 15° C. to 25° C. The resulting mixture was stirred for 30 minutes at a temperature between 15° C. to 25° C. and then the layers were allowed to separate over a period of 30 minutes. The organic layer was collected and to this layer was added dichloromethane (1.5 L). The resulting solution containing the title compound was stored under a nitrogen atmosphere, protected from light, at 0° C. to 5° C. until used in the subsequent reaction.

### Step B—Preparation of 7-{Isopropyl-[1-(4-methoxypyridin-3-ylmethyl)-piperidin-4-yl]amino}heptanal

The temperature of the solution from Step A was adjusted to 5° C. to 15° C. and an aqueous hydrochloric acid solution (prepared by adding 1.4 L of concentrated hydrochloric acid to 14.2 L of deionized water) was added while maintaining the temperature of the reaction mixture below 20° C. The resulting two-phase mixture was stirred at 15° C. to 25° C. for 11 hours. The mixture was allowed to stand without stirring for a period of 30 minutes and the organic layer was removed. To the aqueous layer was added dichloromethane (6 L) and this mixture was stirred for 30 minutes. The layers were then allowed to separate over a period of 30 minutes and the organic layer was removed. This washing procedure of the aqueous layer with dichloromethane was repeated 2 additional times. The resulting aqueous solution containing the title compound was stored under a nitrogen atmosphere, protected from light, at 0° C. to 5° C. until used in the subsequent reaction.

### Step C—Preparation of 4-{N-[7-(3-(S)-1-Carbamoyl-1,1-diphenylmethyl)-pyrrolidin-1-yl)hept-1-yl]-N-(isopropyl)amino}-1-(4-methoxypyrid-3-ylmethyl)piperidine

The temperature of the solution from Step B was adjusted to −5° C. to 5° C. and an aqueous sodium hydroxide solution (prepared by dissolving 230 g of sodium hydroxide in 2.9 L of deionized water) was added while maintaining the temperature of the reaction mixture in the range of −5° C. to 5° C. Acetonitrile (9.3 L) was then added while maintaining the temperature of the reaction mixture in the range of −5° C. to 5° C. (S)-3-(1-Carbamoyl-1,1-diphenylmethyl)pyrrolidine (988 g, 3.52 mol) was then added and the resulting mixture as stirred at −5° C. to 5° C. for 1 hour. Sodium triacetoxyborohydride (853 g, 4.02 mol) was then added in 7 equal portions over a period of 1 hour while maintaining the temperature of the reaction mixture between −5° C. to 5° C. The reaction mixture was then stirred at 0° C. to 5° C. for 4.25 hours. Concentrated hydrochloric acid (8.2 L) was then added to the reaction mixture until the pH was in the range of from 2 to 3 while maintaining the temperature below 20° C. MTBE (9.8 L) was then added and the resulting mixture was stirred for 45 minutes at 15° C. to 25° C. The mixture was allowed to stand without stirring for a period of 30 minutes and the aqueous layer was separated. This washing procedure of the aqueous layer with MTBE was repeated and then MTBE (19.4 L) was added to the aqueous layer. An aqueous sodium hydroxide solution (prepared by dissolving 910 g of sodium hydroxide in 5.7 L of deionized water) was added until the pH of the aqueous layer was 11 to 12 while maintaining the temperature below 20° C. This mixture was stirred for 30 minutes at 15° C. to 25° C. The layers were then allowed to separate over a period of 30 minutes and the layers were separated. To the organic layer was added an aqueous solution of potassium carbonate and sodium metabisulfate (prepared by dissolving 970 g of potassium carbonate and 970 g of sodium metabisulfate in 19.4 L of deionized water) and the resulting mixture was stirred for 3 hours at 15° C. to 25° C. The mixture was allowed to stand without stirring for a period of 30 minutes and the layers were separated. To the organic layer was added an aqueous solution of sodium bicarbonate (prepared by dissolving 1.4 kg of sodium bicarbonate in 15 L of deionized water) and the resulting mixture was stirred for 30 minutes at 15° C. to 25° C. The mixture was allowed to stand without stirring for a period of 30 minutes and then the layers were separated. To the organic layer was added deionized water (15 L) and the resulting mixture was stirred for 30 minutes at 15° C. to 25° C. The mixture was allowed to stand without stirring for a period of 30 minutes and then the layers were separated. To the organic layer was added an aqueous phosphate buffer solution (7.5 L) (prepared by mixing a solution of 2.396 kg of sodium hydrogenphosphate dissolved in 67.5 L of deionized water with a solution of 675 g of sodium dihydrogenphosphate dissolved in 22.5 L of deionzed water) and the resulting mixture was stirred for 30 minutes at 15° C. to 25° C. The mixture was allowed to stand for 10 minutes and then the layers were separated. This procedure was repeated 11 times and then the aqueous layers were combined. To the combined aqueous layers was added MTBE (19.4 L) and then an aqueous sodium hydroxide solution (prepared by dissolving 290 g of sodium hydroxide in 1.8 L of deionized water) was added while maintaining the temperature below 20° C. until the pH of the aqueous layer was 11 to 12. This mixture was stirred for 30 minutes at 15° C. to 25° C. The mixture was allowed to stand without stirring for a period of 30 minutes and the layers were separated. To the organic layer was added deionized water (15 L) and the resulting mixture was stirred for 1.5 hours at 15° C. to 25° C. The mixture was allowed to stand without stirring for a period of 1 hour and then the layers were separated. To the organic layer was added anhydrous magnesium sulfate (3 kg) and the resulting mixture was stirred for 2.25 hours at 15° C. to 30° C. The mixture was then filtered and the filter cake was washed with MTBE (4.5 L). The resulting solution containing the title compound was stored under a nitrogen atmosphere, protected from light, at 0° C. to 5° C. until being used in the subsequent reaction.

### Step D—Preparation of 4-{N-[7-(3-(S)-1-Carbamoyl-1,1-diphenylmethyl)-pyrrolidin-1-yl)hept-1-yl]-N-(isopropyl)amino}-1-(4-methoxypyrid-3-ylmethyl)piperidine

### Naphthalene-1,5-disulfonic Acid Salt

To methanol (6 L) was added naphthalene-1,5-disulfonic acid (641.33 g, 2.22 mol) and the resulting mixture was stirred until the naphthalene-1,5-disulfonic acid completely dissolved. To this solution was added isopropanol (6 L) and the temperature of the resulting mixture was adjusted to 15° C. to 25° C. MTBE (114 L) was added to the solution from Step C and then the solution of naphthalene-1,5-disulfonic acid was added over a period of 2 hours while maintaining the temperature of the reaction mixture at 15° C. to 25° C. Isopropanol (6 L) was then added while maintaining the temperature of the reaction mixture at 15° C. to 25° C. and the resulting mixture was stirred for 12 hours at a temperature in the range of 15° C. to 25° C. The mixture was then cooled to a temperature of 0° C. to 5° C. and stirred for 2 hours. The precipitate which formed was then collected by filtration under nitrogen and the filter cake was washed three times with MTBE (6 L) cooled to 0° C. to 5° C. The precipitate was then dried under vacuum at ambient temperature to provide the title compound (1,452.6 g, 40% overall yield, 99.6% purity by HPLC).

### Example 9 (Comparative)

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Dimethanesulfonic Acid Salt

To a 5 L flask was added 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine (593 g, 0.93 mol) and 1.44 L of absolute ethanol and the mixture was stirred to dissolve the oil. This mixture was then cooled to 0-5° C. and a solution of 142.5 g of methanesulfonic acid (142.5 g, 1.48 mol) in 98 mL of absolute ethanol was added at 5° C. The mixture was stirred at 5-10° C. for 1 h and then it was added to 37.5 L of MTBE slowly and this mixture was stirred for 30 min at 10-15° C. The resulting solid was filtered and dissolved in 5 L of distilled water. The water solution was treated with activated carbon (70 g) and filtered. The filtrate was frozen at −40° C. and lyophilized for 72 hours to give 481 g of the di(methanesulfonic acid) (79% yield, 99.1% purity by HPLC).

### Example 10 (Comparative)

### Synthesis of 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine

### Trimethanesulfonic Acid Salt

A 100 mL Erlenmeyer flask was charged with 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine (3.9 g, 6.1 mmol) and acetonitrile (32 mL) and upon dissolution, water (25 mL) and methanesulfonic acid (1.29 mL, 1.91 g, 19.9 mmol) were added to bring the pH to about 5. The solution was then frozen in a dry ice/acetone bath and lyophilized for 48 h to afford 5.5 g of the tri(methanesulfonic acid) salt as an off-white solid (100% yield; 97.4% purity by HPLC).

Analytical Data: MS m/z 640.5 (MH⁺).

### Example 11

### General Procedures for Preparing Other Comparative Salt Forms

Method A: To an alcoholic solution (methanol, ethanol, or iso-propanol) of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine was added either one, two or three molar equivalents of an acid as either an alcoholic solution or as a solid. The resulting mixture was stirred until homogeneous (if necessary, the mixture was heated to <50° C.). The mixture was then added dropwise to vigorously-stirred MTBE to produce a precipitate (typically a white solid). The precipitate was isolated by filtration, washed with MTBE (3×), and dried on vacuum under nitrogen to afford the comparative salt.

Using this procedure, the following comparative salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine were prepared:
Example 11A: Monosulfuric Acid Salt;
Example 11B: Monotartaric Acid Salt; and
Example 11C: Diorotic Acid Salt.

Method B: To a vigorously-stirred homogeneous solution of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine in isopropanol, isobutanol or ethyl acetate at a temperature ranging from about 22° C. to 50° C. was added a solution (in the same solvent) of either one, two or three molar equivalents of an acid to obtain a white precipitate. The resulting mixture was slowly cooled to 0° C. to 20° C. and the precipitate was isolated by filtration. The precipitate was then washed (3×) with either solvent, MTBE or both and then dried on vacuum under nitrogen to afford the comparative salt.

Using this procedure, the following comparative salts of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine were prepared:
Example 11D: Disalicylic Acid Salt;
Example 11E: Trisalicylic Acid Salt; and
Example 11F: Digentisic Acid Salt.

Method C: Using the procedure of Example 9, i.e., lyophilization, the following comparative salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine was prepared:
Example 11G: Dihydrochloric Acid Salt.

### Example 12

### Method for Determining the Chemical Stability of Salt Forms

The chemical stability of each salt form was evaluated by determining the change in purity of the sample upon storage of the salt form at 40° C.

Prior to storage, each salt form was analyzed by HPLC (Method D) to determine sample purity and in particular, to determine the amount of the following impurities present in the sample:
A. 3-[4-{N-[7-(3-(S)-1-Carbamoyl-1,1-diphenylmethyl)pyrrolidin-1-yl)hept-1-yl]-N-(isopropyl)amino}piperidin-1-ylmethyl]-4-methoxy-1-methylpyridinium Salt (Impurity A);
B. 4-{N-[7-(3-(S)-1-Carbamoyl-1,1-diphenylmethyl)pyrrolidin-1-yl)hept-1-yl]-N-(isopropyl)amino}-1-(4-oxo-1,4-dihydropyrid-3-ylmethyl)piperidine (Impurity B).

Approximately 50-100 mg of the salt form was then placed in two heat sealed low density polyethylene bags. The bags were then placed into a stability chamber that was previously set at 40° C. and 75% relative humidity. After 7 days, the bags were removed and the contents were analyzed by HPLC. The results are shown in Table I.

TABLE I
Chemical Stability of Salt Forms

| | | % Change in | % Change in | % Change in |
|---|---|---|---|---|
| Ex. | Salt Form | Purity of Salt | Impurity A | Impurity B |
| 6 | Free Base | 1.2 | <0.1 | <0.1 |
| 7 | Naphthalene- | 0.4 | <0.1 | <0.1 |
| | 1,5-disulfonic | | | |
| | Acid Salt | | | |
| 9 | Dimethanesulfonic | 0.9 | <0.1 | <0.1 |
| | Acid Salt | | | |
| 10 | Trimethanesulfonic | 16.4 | 9.41 | 6.59 |
| | Acid Salt | | | |
| 11A | Monosulfuric Acid | 0.8 | <0.1 | 0.11 |
| | Salt | | | |
| 11B | Monotartaric Acid Salt | 0.8 | 0.26 | 0.35 |
| 11C | Diorotic Acid Salt | 1.1 | 0.45 | 0.49 |
| 11D | Disalicylic Acid Salt | 0.7 | 0.17 | 0.19 |
| 11E | Trisalicylic Acid Salt | 2.7 | 1.22 | 1.06 |
| 11F | Digentisic Acid Salt | 0.4 | 0.21 | 0.23 |
| 11G | Dihydrochloric Acid | 3.6 | 1.16 | 0.79 |
| | Salt | | | |

The data in Table I demonstrate that the naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine had excellent chemical stability. In contrast, the other salt forms tested had a greater change in purity and/or generated higher amounts of Impurities A or B or both.

### Example 13

### Method for Determining the Physical Stability of Salt Forms

The physical stability of certain salt forms was evaluated by determining any changes in appearance of the sample upon storage of the salt form at 30° C. and 60% relative humidity.

To an open vial was added 50-100 mg of each salt form. The open vials were placed in a stability chamber that was previously set at 30° C. and 60% relative humidity. At periodic intervals, the appearance of each salt form was compared to its starting appearance and any differences were recorded. The results are shown in Table II.

TABLE II
Physical Stability of Salt Forms

| Ex. | Salt Form | Day 0 | Day 1 | Day 2 | Day 15 |
|---|---|---|---|---|---|
| 7 | Naphthalene-1,5- | flowing | flowing | flowing | flowing white |
| | disulfonic Acid Salt | white | white | white | powder; some |
| | | powder | powder | powder | slight |
| | | | | | agglomeration |
| 11A | Monosulfuric Acid | flowing | clear | clear | clear |
| | Salt | white | deliquesced | deliquesced | deliquesced |
| | | powder | | | |
| 11B | Monotartaric Acid | flowing | white | white | white |
| | Salt | white | deliquesced | deliquesced | deliquesced |
| | | powder | | | |
| 11C | Diorotic Acid Salt | flowing | tacky | tacky | tacky |
| | | white | agglomerated | agglomerated | agglomerated |
| | | powder | solid | solid | solid |
| 11F | Digentisic Acid Salt | flowing | flowing | white | white powder, |
| | | white | white | powder, | slightly tacky |
| | | powder | powder | slightly tacky | |

The data in Table II demonstrate that, of the salt forms tested, only the naphthalene-1,5-disulfonic acid salt remained a free flowing powder after 15 days at 30° C. and 60% relative humidity. In contrast, the other salt forms tested were either deliquescent or did not remain free flowing.

### Example 14

### Method for Determining the Physical Stability of Pharmaceutical Formulations

The physical stability of encapsulated pharmaceutical formulations containing certain salt forms was evaluated by determining any changes in appearance of the pharmaceutical formulation upon storage.

Capsules containing a 1:1 mixture (wt/wt) of the salt form and microcrystalline cellulose (Avecil) were stored under of the following conditions:
(1) Capsules were placed inside an open container within a stability chamber that was previously set to 25° C. and 60% relative humidity; or
(2) Capsules were placed inside an open container on the bench top alongside a thermometer and hygrometer.

At periodic intervals, the appearance of each formulation was compared to its starting appearance and any differences were recorded. The results are shown in Table III.

TABLE III
Physical Stability of Pharmaceutical Formulations
At 25° C. and 60% Relative Humidity

| Ex. | Salt Form | 0 Hours | 6 Hours | 30 Hours |
|---|---|---|---|---|
| 7 | Naphthalene- | flowing white | flowing white | flowing white |
| | 1,5-disulfonic | powder | powder | powder |
| | Acid Salt | | | |
| 11B | Monotartaric | flowing white | non-flowing | non-flowing |
| | Acid Salt | powder | granular cake | granular cake |

At Ambient Temperature (20-24° C.) and Relative Humidity (30-44%)

| Ex. | Salt Form | 0 Hours | 6 Hours | 48 Hours |
|---|---|---|---|---|
| 7 | Naphthalene- | flowing white | flowing white | flowing white |
| | 1,5-disulfonic | powder | powder | powder |
| | Acid Salt | | | |
| 11B | Monotartaric | flowing white | flowing white | non-flowing |
| | Acid Salt | powder | powder | granular cake |

The data in Table III demonstrate that the pharmaceutical formulation containing the naphthalene-1,5-disulfonic acid salt remained a free flowing powder at both 25° C. and 60% relative humidity, and at ambient temperature and relative humidity. In contrast, the pharmaceutical formulation containing the monotartaric acid salt formed a non-flowing granular cake under the same conditions.

### Example 15

### Radioligand Binding Assay

### A. Membrane Preparation from Cells Expressing hM₁, hM₂, hM₃ and hM₄ Muscarinic Receptor Subtypes

CHO (Chinese hamster ovary) cell lines stabilely expressing cloned human hM₁, hM₂, hM₃ and hM₄ muscarinic receptor subtypes, respectively, were grown to near confluency in medium consisting of HAM's F-12 supplemented with 10% FBS (Fetal Bovine Serum) and 250 μg/mL Geneticin. The cells were grown in a 5% CO₂, 37° C. incubator and lifted with dPBS+2 mM EDTA. Cells were collected by 5 minute centrifugation at 650×g, and cell pellets were either stored frozen at −80° C. or membranes were prepared immediately. For membrane preparation, cell pellets were resuspended in lysis buffer and homogenized with a Polytron PT-2100 tissue disrupter (Kinematica AG; 20 seconds×2 bursts). Crude membranes were centrifuged at 40,000×g for 15 minutes at 4° C. The membrane pellet was then resuspended with resuspension buffer and homogenized again with the Polytron tissue disrupter. Protein concentration of the membrane suspension was determined by the method of Lowry, O. et al., (1951) Journal of Biochemistry: 193, 265. Membranes were stored frozen in aliquots at −80° C.

Aliquots of prepared hM₅ receptor membranes were purchased directly from Perkin Elmer and stored at −80° C. until use.

### B. Radioligand Binding Assay on Muscarinic Receptor Subtypes hM₁, hM₂, hM₃, hM₄ and hM₅

Radioligand binding assays were performed in 96-well microtiter plates in a total assay volume of 100 μL. Membranes containing each of the respective muscarinic subtypes were diluted in assay buffer to the following specific target protein concentrations (μg/well): 10 μg for hM₁, 10-15 μg for hM₂, 10-20 μg for hM₃, 18-20 μg for hM₄, and 10-12 μg for hM₅. The membranes were briefly homogenized using a Polytron tissue disruptor (10 seconds) prior to assay plate addition. Saturation binding studies for determining K_{D} values of the radioligand were performed using l-[N-methyl-³H]scopolamine methyl chloride ([³H]NMS) (TRK666, 84.0 Ci/mmol, Amersham Pharmacia Biotech, Buckinghamshire, England) at concentrations ranging from 0.001 nM to 20 nM. Displacement assays for determination of Kᵢ values of a test compound were performed with [³H]NMS at 1 nM and eleven different test compound concentrations. The test compound was initially dissolved to a concentration of 400 μM in dilution buffer and then serially diluted 5× with dilution buffer to final concentrations ranging from 10 μM to 100 μM. The addition order and volumes to the assay plates were as follows: 25 μL radioligand, 25 μL diluted test compound, and 50 μL membranes. Assay plates were incubated for 60 minutes at 37° C. Binding reactions were terminated by rapid filtration over GF/B glass fiber filter plates (PerkinElmer Inc., Wellesley, Mass.) pre-treated in 1% BSA. Filter plates were rinsed three times with wash buffer (10 mM HEPES) to remove unbound radioactivity. Plates were air dried, and 50 μL Microscint-20 liquid scintillation fluid (PerkinElmer Inc., Wellesley, Mass.) was added to each well. The plates were then counted in a PerkinElmer Topcount liquid scintillation counter (PerkinElmer Inc., Wellesley, Mass.). Binding data were analyzed by nonlinear regression analysis with the GraphPad Prism Software package (GraphPad Software, Inc., San Diego, Calif.) using the one-site competition model. A Kᵢ value for the test compound was calculated from the observed IC₅₀ value and the K_{D} value of the radioligand using the Cheng-Prusoff equation (Cheng Y; Prusoff W H. (1973) Biochemical Pharmacology, 22(23):3099-108). The Kᵢ value was converted to a pKᵢ value to determine the geometric mean and 95% confidence intervals. These summary statistics were then converted back to a Kᵢ value for data reporting.

Test compounds having a lower Kᵢ value in this assay have a higher binding affinity for the muscarinic receptor. The compound of formula I had a Kᵢ value for hM₂ in this assay of less than 1 nM and an hM₃/hM₂ ratio greater than 40. Thus, the compound of formula I was found to bind potently to the hM₂ receptor subtype in this assay and to have a higher binding affinity for the hM₂ receptor subtype relative to the hM₃ receptor subtype.

### Example 16

### In Vivo Rat Bladder Assay

Female Sprague-Dawley rats (Harlan, Indianapolis, Ind.) weighing 200 to 300 g were anesthetized with urethane (1.5 g/kg, s.c., Sigma, St. Louis, Mo.), with a supplement of 0.25 g/kg, s.c. urethane as needed. Urethane was administered at a concentration of 0.25 g/mL.

Rats were prepared for surgery by shaving the neck and abdomen and cleansing with ethanol wipes. First, an incision was made on the ventral surface. An intravenous catheter was placed by isolating and ligating the femoral vein. A small incision was made in the vein proximal to the ligation through which a catheter (micro-Renathane tubing, 0.30 mm ID×0.64 mm OD, Becton Dickinson, Sparks, Md.) filled with D5W was inserted and secured in place with 4.0 silk suture thread (Ethicon, Johnson and Johnson, Somerville, N.J.). Similarly, a catheter was inserted into the femoral artery for the measurement of cardiovascular parameters. A tracheotomy was performed by isolating the trachea and placing a small hole between two tracheal rings. PE 205 tubing (1.57 mm ID×2.08 mm OD, Becton Dickinson, Sparks, Md.) was inserted into the trachea toward the lungs. The neck incision was closed with 9 mm wound clips leaving the catheters and distal end of the trachea tube exposed.

Subsequently, a 3 cm midline sagital incision in the skin and muscle layers of the lower abdomen was made. The bladder and ureters were isolated and exposed by means of tissue forceps. The ureters were ligated and severed distal to the bladder. The bladder was cannulated with PE50 tubing (0.58 mm ID×0.965 mm OD, Becton Dickinson, Sparks, Md.) via the urethra. The cannula was attached to a micro infusion pump to allow infusion of saline into the bladder through a pressure transducer (Argon, Athen, Tex.). The cannula was secured in place using a purse string suture (4.0 silk suture). To ensure a tight seal, the cannula was tied in place around the external urethral orifice with 2.0 silk suture thread. After the bladder was placed back into the peritoneal cavity, the bladder was manually voided allowing the contents to flow out until the bladder was empty. The incision was closed with 9 mm wound clips.

After the surgical preparation, the bladder was filled with saline at a constant rate of 200 μL/min for 5 minutes or until bladder pressure averaged over 30 mm Hg. Subsequently, the bladder was filled with a maintenance infusion of 5 μL/min. When rhythmic volume-induced bladder contractions (VIBC's) were observed, the maintenance infusion was adjusted 2 to 5 μL/min. Only rats demonstrating rhythmic bladder contractions of similar peak height were used in the experiment. Animals not demonstrating this profile within 60 minutes were euthanized by CO₂ asphyxiation.

Once stable rhythmic VIBC's were observed for at least 30 minutes during the maintenance infusion, vehicle (D5W) was infused intravenously (1 mL/kg) and changes in VIBC amplitude (VIBC_{Amp}) were recorded for 15 minutes. Thereafter, an intravenous dose of the test compound was administered and changes in VIBC_{Amp} were recorded for 15 minutes. Atropine (0.1 mg/kg) was then administered intravenously (1 mL/kg) as a positive control and VIBC_{Amp} and data was recorded for an additional 15 minutes. At least four doses of each test compound at half log increments were tested in this model.

Alternatively, after the vehicle, increasing cumulative intravenous doses of the test compound were administered at 15 minute intervals (1 mL/kg) and changes in VIBC_{Amp} were recorded for 15 minutes. At least 4 doses of test compound were administered at half log increments.

The average VIBC_{Amp} during the 5-15 minute period after test compound and atropine was determined and subtracted from the average VIBC_{Amp} during the 5-15 minute post-vehicle period to obtain the test compound or atropine-induced change in VIBC_{Amp}. The inhibitory effects of the test compound were normalized to the atropine response and the resulting dose-response curves were fitted with a four parameter logistic equation to obtain estimates of ID50 (dose required to produce 50% of the maximal response).

Test compounds having a lower ID₅₀ value in this assay are more effective for reducing peak bladder contraction pressure. In this assay, the compound of formula I had an ID₅₀ value of less than or equal to about 0.1 mg/kg.

While the present invention has been described with reference to specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. Additionally, to the extent permitted by applicable patent statutes and regulations, all publications, patents, and patent documents cited herein are incorporated by reference herein in their entirety to the same extent as if they had been individually incorporated by reference.

1. A naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or a solvate thereof; wherein the salt has a molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranging from about 0.7 to about 1.1.

2. The salt of claim 1, wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.8 to about 1.05.

3. The salt of claim 1, wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.9 to about 1.

4. The salt of any one of claims 1 to 3, wherein the salt is an amorphous powder.

5. The salt of any one of claims 1 to 3, wherein the salt is characterized by an FTIR spectrum having peaks at about 1671.7, 1593.5, 1497.6, 1291.2, 1220.9, 1180.3 and 1030.1 cm⁻¹.

6. The salt of any one of claims 1 to 3, wherein the purity of the salt is greater than about 98% by weight.

7. The salt of any one of claims 1 to 3, wherein the salt is substantially free of a 3-[4-{N-[7-(3-(S)-1-carbamoyl-1,1-diphenylmethyl)pyrrolidin-1-yl)hept-1-yl]-N-(isopropyl)amino}piperidin-1-ylmethyl]-4-methoxy-1-methylpyridinium salt.

8. 4-{N-[7-(3-(S)-1-Carbamoyl-1,1-diphenylmethyl)pyrrolidin-1-yl)hept-1-yl]-N-(isopropyl)amino}-1-(4-methoxypyrid-3-ylmethyl)piperidine mononaphthalene-1,5-disulfonic acid salt.

9. A pharmaceutical composition comprising a pharmaceutically-acceptable carrier and a therapeutically effective amount of a naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or a solvate thereof; wherein the salt has a molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranging from about 0.7 to about 1.1.

10. The pharmaceutical composition of claim 9, wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.8 to about 1.05.

11. The pharmaceutical composition of claim 9, wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.9 to about 1.

12. The pharmaceutical composition of any one of claims 9 to 11, wherein the composition is in a unit dosage form.

13. The pharmaceutical composition of claim 12, wherein the composition is a tablet, capsule or pill.

14. A method for treating a medical condition alleviated by treatment with a muscarinic receptor antagonist in a mammal, the method comprising administering to the mammal a therapeutically effective amount of a pharmaceutical composition comprising a pharmaceutically-acceptable carrier and a naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or a solvate thereof; wherein the salt has a molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranging from about 0.7 to about 1.1.

15. A method for treating overactive bladder in a mammal, the method comprising administering to the mammal a therapeutically effective amount of a pharmaceutical composition comprising a pharmaceutically-acceptable carrier and a naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or a solvate thereof; wherein the salt has a molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranging from about 0.7 to about 1.1.

16. The method of claim 14 or 15, wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.8 to about 1.05.

17. The method of claim 14 or 15, wherein the molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranges from about 0.9 to about 1.

18. A process for preparing a naphthalene-1,5-disulfonic acid salt of 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or a solvate thereof; wherein the salt has a molar ratio of naphthalene-1,5-disulfonic acid to 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine ranging from about 0.7 to about 1.1; the process comprising contacting 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine with about 0.7 to about 1.1 molar equivalents of 1,5-naphthalenedisulfonic acid or a hydrate thereof.

19. The process of claim 18, wherein the process further comprises the step of forming an amorphous powder of naphthalene-1,5-disulfonic acid salt of the 4-{N-[7-(3-(S)-1-carbamoyl-1,1-diphenylmethyl)pyrrolidin-1-yl)hept-1-yl]-N-(isopropyl)amino}-1-(4-methoxypyrid-3-ylmethyl)piperidine or solvate thereof.

20. The product prepared by the process of claims 18 or 19.
